(19) United States
(12) Patent Application Publication
(10) Pub. No.: US 2026/0363646 A1
(43) Pub. Date: Nov. 19, 2026

(54) INFORMATION PROCESSING SYSTEM, INFORMATION PROCESSING DEVICE, CONTROL METHOD, AND COMPUTER-READABLE STORAGE MEDIUM

(71) Applicant: SEKISUI CHEMICAL CO., LTD., Osaka (JP)

(72) Inventors: Kouzou NAKAMURA, Osaka (JP); Yasuyuki SHIRASAKA, Osaka (JP); Jiyunichi MATSUZAKI, Ibaraki (JP); Akihiro UENISHI, Ibaraki (JP); Ryousuke SUDO, Osaka (JP)

(73) Assignee: SEKISUI CHEMICAL CO., LTD., Osaka (JP)

(21) Appl. No.: 19/137,092

(22) PCT Filed: Dec. 1, 2023
(86) PCT No.: PCT/JP2023/043150

§ 371 Date: Jun. 9, 2025

(30) Foreign Application Priority Data
Dec. 12, 2022 (JP) 2022-198128
Mar. 30, 2023 (JP) 2023-056099

(51) Int. Cl. A61B 7/00; A61B 5/00; G16H 40/67

(52) U.S. Cl. CPC A61B 7/008; A61B 5/4238; A61B 5/4255; A61B 5/4561; A61B 5/4809; A61B 5/6891; A61B 5/6892; A61B 5/7267; G16H 40/67

(57) ABSTRACT

A peristaltic sound signal which indicates a peristaltic sound and which corresponds to the posture of a subject is extracted accurately. An information processing system (1) includes: a sensor (11) that is disposed at a given position at which the sensor does not touch a subject and that detects vibrations which have been generated from the subject; and a peristaltic sound generation determining section (124a, 424a) that determines whether or not a peristaltic sound has been generated in the stomach and intestines of the subject, on the basis of a detection signal outputted from the sensor (11).

### TECHNICAL FIELD

The present invention relates to an information processing system etc. for detecting vibrations generated by a subject.

### BACKGROUND ART

A variety of techniques for measuring peristalses of the stomach and intestines of a subject have been developed. For example, Patent Literature 1 discloses an intestinal sound measuring apparatus for grasping the condition of the intestines on the basis of a peristaltic sound in the intestines.

### CITATION LIST

### Patent Literature

### [Patent Literature 1]

Japanese Patent Application Publication Tokukai No. 2016-36637

### SUMMARY OF INVENTION

### Technical Problem

A technique of attaching or touching a sensor to a subject and measuring a peristaltic sound in the stomach and intestines may make the subject feel discomfort, and is therefore not suitable for long-term monitoring and constant monitoring.

### Solution to Problem

An information processing system in accordance with an aspect of the present disclosure includes: a sensor that is disposed at a given position at which the sensor does not touch a subject and that detects vibrations which have been generated from the subject; and a peristaltic sound generation determining section that determines whether or not a peristaltic sound has been generated in the stomach and intestines of the subject, on the basis of a detection signal outputted from the sensor.

An information processing system in accordance with an aspect of the present disclosure includes: a sensor that is disposed at a given position at which the sensor does not touch a subject and that detects vibrations which have been generated from the subject; and a signal extracting section that extracts a peristaltic sound signal indicating a peristaltic sound in the stomach and intestines of the subject, on the basis of a detection signal outputted from the sensor.

An information processing system in accordance with an aspect of the present disclosure includes: a sensor that is disposed at a given position at which the sensor does not touch a subject and that detects vibrations which have been generated from the subject; a signal extracting section that extracts, from a detection signal outputted from the sensor, a peristaltic sound signal indicating a peristaltic sound in the stomach and intestines of the subject; and a posture determining section that determines a posture of the subject on the basis of the detection signal, wherein the signal extracting section extracts the peristaltic sound signal corresponding to the posture which has been determined.

An information processing system in accordance with an aspect of the present disclosure includes: a sensor that is disposed at a given position at which the sensor does not touch a subject and that detects vibrations which have been generated from the subject; a signal extracting section that extracts, from a detection signal outputted from the sensor, a heart sound signal indicating a heart sound of the subject and a peristaltic sound signal indicating a peristaltic sound in the stomach and intestines of the subject; and a posture determining section that determines a posture of the subject on the basis of the heart sound signal which has been extracted, wherein the signal extracting section extracts the peristaltic sound signal having a signal strength corresponding to the posture which has been determined.

An information processing system in accordance with an aspect of the present disclosure includes: a sensor that is disposed at a given position at which the sensor does not touch a subject and that detects vibrations which have been generated from the subject; a signal extracting section that extracts, from a detection signal outputted from the sensor, a peristaltic sound signal indicating a peristaltic sound in the stomach and intestines of the subject; a posture determining section that determines a posture of the subject; and a generation position estimating section that estimates a position of generation of the peristaltic sound indicated by the peristaltic sound signal, on the basis of the peristaltic sound signal which has been extracted and the posture which has been determined.

An information processing apparatus in accordance with an aspect of the present disclosure includes: a sensor that is disposed at a given position at which the sensor does not touch a subject and that detects vibrations which have been generated from the subject; and a signal extracting section that extracts, from a detection signal outputted from the sensor, a peristaltic sound signal indicating a peristaltic sound in the stomach and intestines of the subject.

A control method in accordance with an aspect of the present disclosure is a control method which is carried out by one or more information processing apparatuses, the method including: an outputting step of outputting a detection signal from a sensor that is disposed at a given position at which the sensor does not touch a subject and that detects vibrations which have been generated from the subject; an extracting step of extracting, from the detection signal which has been outputted, a peristaltic sound signal indicating a peristaltic sound in the stomach and intestines of the subject; and a determining step of determining a posture of the subject on the basis of the detection signal.

The information processing systems and the information processing apparatus in accordance with the above aspects may be each implemented by a computer. In this case, the present invention also encompasses, in its scope, (i) a control program for each of the information processing systems and the information processing apparatus, the control program causing each of the information processing systems and the information processing apparatus to be implemented by the computer, by causing the computer to operate as each section (software element) included in each of the information processing system and the information processing apparatus and (ii) a computer-readable recording medium in which the control program is stored.

### Advantageous Effects of Invention

An aspect of the present disclosure makes it possible to accurately grasp peristalses of the stomach and intestines of a subject with a simple configuration and without making the subject feel discomfort.

### BRIEF DESCRIPTION OF DRAWINGS

FIG. 1 illustrates sonographs each indicating a peristaltic sound signal included in a detection signal.

FIG. 2 is a conceptual diagram illustrating an example configuration of an information processing system.

FIG. 3 illustrates example schematic configurations of an information processing apparatus of the information processing system.

FIG. 4 illustrates a different example schematic configuration of the information processing apparatus of the information processing system.

FIG. 5 illustrates another example schematic configuration of the information processing apparatus of the information processing system.

FIG. 6 illustrates the frequency characteristic and signal strength of a peristaltic sound signal.

FIG. 7 is a functional block diagram illustrating an example configuration of the information processing system.

FIG. 8 is a flowchart illustrating an example flow of processes carried out by the information processing system.

FIG. 9 is a functional block diagram illustrating an example configuration of an information processing system.

FIG. 10 is a flowchart illustrating an example flow of processes carried out by the information processing system.

FIG. 11 is a functional block diagram illustrating an example configuration of an information processing system.

FIG. 12 illustrates a feature signal included in a detection signal.

FIG. 13 is a flowchart illustrating an example flow of processes carried out by the information processing system.

FIG. 14 is a functional block diagram illustrating an example configuration of an information processing system.

FIG. 15 is a flowchart illustrating an example flow of processes carried out by the information processing system.

FIG. 16 is a functional block diagram illustrating an example configuration of an information processing system.

FIG. 17 is a flowchart illustrating an example flow of processes carried out by the information processing system.

FIG. 18 is a functional block diagram illustrating a different example configuration of an information processing system.

FIG. 19 is a flowchart illustrating an example flow of processes carried out by the information processing system.

FIG. 20 is a functional block diagram illustrating an example configuration of an information processing system.

FIG. 21 is a flowchart illustrating an example flow of processes carried out by the information processing system.

FIG. 22 is a functional block diagram illustrating an example configuration of an information processing system.

FIG. 23 is a flowchart illustrating an example flow of processes carried out by the information processing system.

FIG. 24 is a functional block diagram illustrating an example configuration of an information processing system.

FIG. 25 is a flowchart illustrating an example flow of processes carried out by the information processing system.

FIG. 26 is a functional block diagram illustrating an example configuration of an information processing system.

FIG. 27 is a flowchart illustrating an example flow of processes carried out by the information processing system.

FIG. 28 is a functional block diagram illustrating an example configuration of an information processing system.

FIG. 29 is a flowchart illustrating an example flow of processes carried out by the information processing system.

FIG. 30 is a functional block diagram illustrating an example configuration of an information processing system.

FIG. 31 is a flowchart illustrating an example flow of processes carried out by the information processing system.

FIG. 32 is a functional block diagram illustrating an example configuration of an information processing system.

FIG. 33 is a flowchart illustrating an example flow of processes carried out by the information processing system.

FIG. 34 is a functional block diagram illustrating an example configuration of an information processing system.

FIG. 35 is a flowchart illustrating an example flow of processes carried out by the information processing system.

FIG. 36 is a functional block diagram illustrating an example configuration of an information processing system.

FIG. 37 is a flowchart illustrating an example flow of processes carried out by the information processing system.

FIG. 38 is a functional block diagram illustrating an example configuration of an information processing system.

FIG. 39 is a flowchart illustrating an example flow of processes carried out by the information processing system.

FIG. 40 is a functional block diagram illustrating an example configuration of an information processing system.

FIG. 41 is a flowchart illustrating an example flow of processes carried out by the information processing system.

FIG. 42 is a functional block diagram illustrating an example configuration of an information processing system.

FIG. 43 is a flowchart illustrating an example flow of processes carried out by the information processing system.

FIG. 44 illustrates an example display screen displayed by a display section of the information processing system.

FIG. 45 is a schematic view for explaining a model of a neuron.

FIG. 46 is for explaining a neural network.

### DESCRIPTION OF EMBODIMENTS

### Embodiment 1

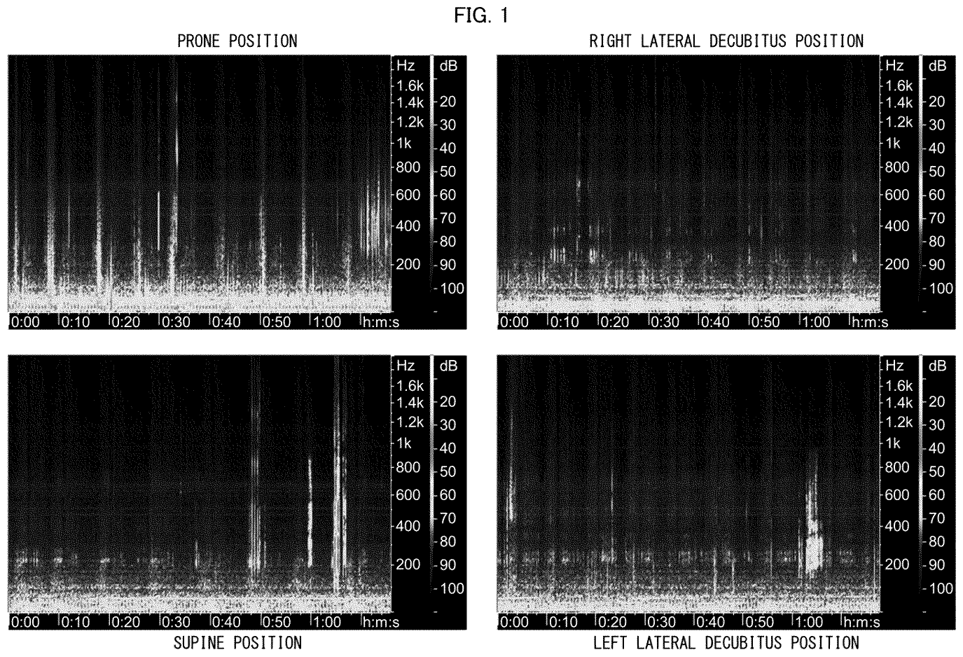

The following description will discuss an embodiment of the present invention with reference to FIGS. 1 to 13.

### (Outline of Information Processing System 100)

The outline of an information processing system 100 in accordance with Embodiment 1 of the present disclosure is described with reference to FIG. 1. FIG. 1 illustrates sonographs each indicating a peristaltic sound signal included in a detection signal. A “sonograph” is a graph showing a sound or a vibration. In each of the sonographs, the horizontal axis indicates time, the vertical axis indicates a frequency, and the colors (light and shade) indicate an energy distribution (signal strength).

The information processing system 100 is a system in which a peristaltic sound signal corresponding to the posture of a subject W1 is extracted on the basis of a detection signal outputted from a sensor that detects, at a position at which the sensor does not touch the subject W1, vibrations which have been generated from the subject W1.

In this specification, a “subject” is typically a person who needs to be monitored by a medical expert W2 or others, such as a patient lying on a bed or the like. A “heart sound signal” is a signal indicating a sound of a heartbeat of a subject. A “heartbeat-related ballistic motion signal” is a signal indicating all vibrations which are derived from a heartbeat and which are generated on the body surface.

A conventional apparatus which measures a peristaltic sound in the stomach and intestines of a subject is used in a state in which a sensor is attached or touched to the subject. This may make the subject feel discomfort. Therefore, the conventional apparatus is not suitable for long-term monitoring or constant monitoring. Note that, in a case where a peristaltic sound is measured with use of a sensor which does not directly touch a subject, it may not be possible to appropriately measure the peristaltic sound, depending on the posture (right lateral decubitus position, left lateral decubitus position, supine position, prone position, and sitting position) of the subject.

In the present disclosure, a first meaning of the phrase “does not directly touch a subject” is “does not touch the skin of a subject”. In the present invention, a second meaning of the phrase “does not directly touch a subject” is “does not touch the skin of a subject and does not touch the subject via a piece of clothing worn by the subject”. For example, the sensor is disposed between a bed on which a subject lies and a mattress which is placed on the bed, between a bed sheet which is placed on the mattress and the mattress, in the mattress, or at other positions.

The inventors of the present invention found that a peristaltic sound signal which is included in a detection signal outputted from a sensor when the sensor is disposed at a given position has a signal strength which varies depending on the posture of a subject W1. FIG. 1 illustrates sonographs each indicating a peristaltic sound signal which has been extracted from a detection signal outputted from a sensor when a subject W1 takes various postures. The upper left graph in FIG. 1 is a sonograph indicating the peristaltic sound signal in a case where the sensor is disposed on the abdomen side of the subject W1 lying in a prone position. The upper right graph in FIG. 1 is a sonograph indicating the peristaltic sound signal in a case where the sensor is disposed on the right side of the trunk of the subject W1 lying in a right lateral decubitus position. The lower left graph in FIG. 1 is a sonograph indicating the peristaltic sound signal in a case where the sensor is disposed on the back side of the subject W1 lying in a supine position. The lower right graph in FIG. 1 is a sonograph indicating the peristaltic sound signal in a case where the sensor is disposed on the left side of the trunk of the subject W1 lying in a left lateral decubitus position. A comparison of the sonographs in FIG. 1 indicates differences in light and shade, which indicate an energy density (signal strength). Thus, the peristaltic sound signal has a signal strength which varies depending on the posture of the subject W1.

Thus, the information processing system 100 is configured such that a peristaltic sound signal which indicates a peristaltic sound and which corresponds to the posture of a subject W1 is extracted from a detection signal obtained by detecting vibrations which have been generated by the subject W1. This realizes an information processing system which makes it possible to measure a peristaltic sound in the stomach and intestines of a subject W1, regardless of the posture of the subject W1 during measurement. Moreover, since the information processing system 100 is configured such that, during measurement, the sensor detects the vibrations at a given position at which the sensor does not touch the subject W1, it is possible to reduce the possibility that the subject feels discomfort. Furthermore, since the information processing system 100 is configured such that the peristaltic sound signal corresponding to the posture of the subject W1 is extracted, it is possible to carry out appropriate measurement, regardless of the posture of the subject W1. Since, as described above, the information processing system 100 is configured such that the peristaltic sound corresponding to the posture of the subject W1 is measured without making the subject W1 feel discomfort, it is possible to realize highly accurate and long-period-of-time constant monitoring.

### (Configuration of Information Processing System 100)

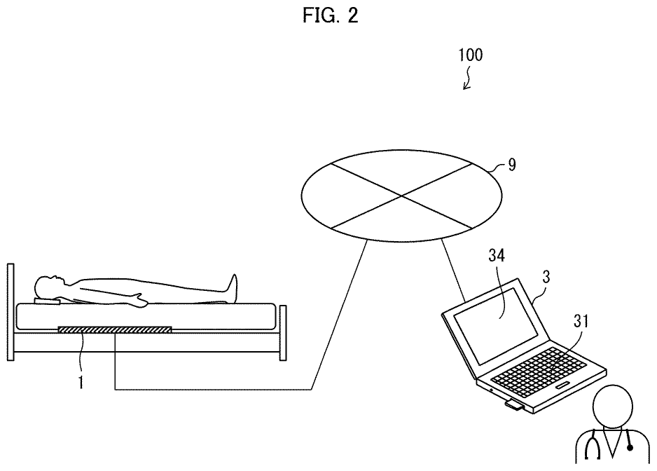

A schematic configuration of the information processing system 100 is described with reference to FIG. 2. FIG. 2 is a conceptual diagram illustrating an example configuration of the information processing system 100.

The information processing system 100 includes a computer which operates as one or more information processing apparatuses. As an example, as illustrated in FIG. 2, the information processing system 100 may include an information processing apparatus 1 and a communication apparatus 3. The number of information processing apparatuses 1 and the number of communication apparatuses 3 may be each one or may be two or more.

The information processing apparatus 1 is a computer which is installed in the vicinity of a subject W1. The information processing apparatus 1 extracts a peristaltic sound signal corresponding to the posture of the subject W1 on the basis of vibrations generated from the subject W1, and outputs the peristaltic sound signal to an external apparatus.

A typical example of the external apparatus to which the peristaltic sound signal is outputted is the communication apparatus 3. The communication apparatus 3 is typically a computer, a smartphone, a tablet terminal, or the like which is used by a medical expert W2, and is installed in, for example, a nurses' station. The information processing apparatus 1 and the communication apparatus 3 may be directly connected to each other. A communication network 9 is not limited to any form, but may be a local area network (LAN) or may be the Internet. Note, however, that, since real-time monitoring of the subject W1 is often required, the information processing apparatus 1 and the communication apparatus 3 are preferably connected to each other such that little delay occurs in time from when the information processing apparatus 1 extracts the peristaltic sound signal until when the communication apparatus 3 displays information indicating the peristaltic sound signal.

The information processing system 100 may include a server apparatus (not illustrated) that is communicatively connected to the information processing apparatus 1 and the communication apparatus 3, in addition to the information processing apparatus 1 and the communication apparatus 3. For example, the server apparatus may be configured to, for each subject, store therein and manage information transmitted from a plurality of information processing apparatuses 1. In this case, the medical expert W2 may access information which pertains to the subject W1 and which is managed by the server apparatus, with use of the communication apparatus 3.

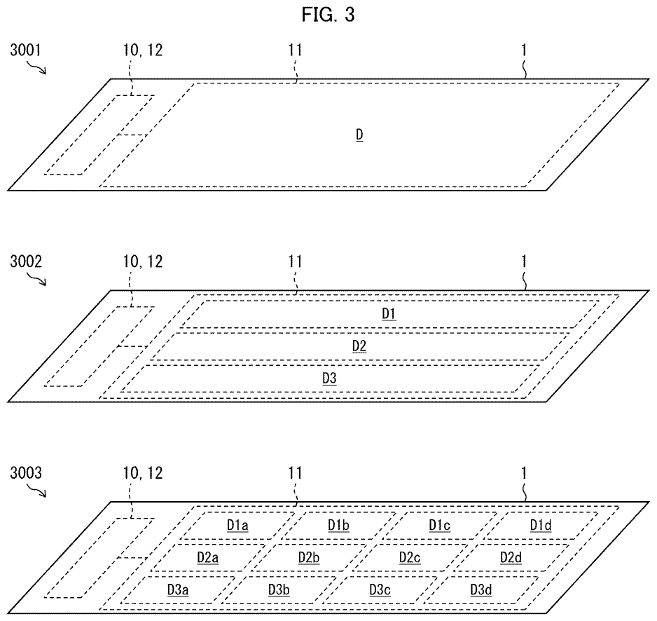

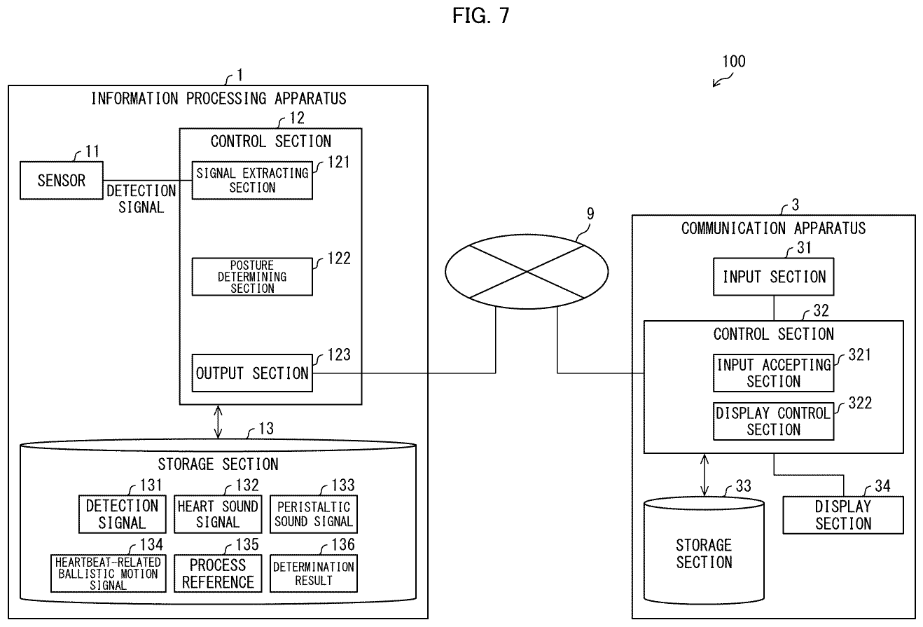

(Configuration of Information Processing Apparatus 1) A schematic configuration of the information processing apparatus 1 is described with use of FIG. 7 with reference to FIGS. 3 to 6. FIG. 7 is a functional block diagram illustrating an example configuration of the information processing system 100.

The information processing apparatus 1 detects, at a given position at which the information processing apparatus 1 does not touch the subject W1, vibrations which have been generated from the subject W1, and extracts a peristaltic sound signal corresponding to the posture of the subject W1 from a signal which has been detected (detection signal). As illustrated in FIG. 7, the information processing apparatus 1 includes, as an example, a sensor 11, a control section 12 which includes a signal extracting section 121, a posture determining section 122, and an output section 123, and a storage section 13. The sensor 11 may not be included in the information processing apparatus 1, and may be provided outside the information processing apparatus 1 and connected to the information processing apparatus 1.

### [Sensor 11]

The sensor 11 is a noncontact (noninvasive) sensor which can detect, at a given position at which the sensor does not touch the subject W1, vibrations which have been generated from the subject W1. The sensor 11 is not limited to a particular type. For example, the sensor 11 is preferably a sensor that detects vibrations, and more preferably a piezoelectric sensor. Adopting a piezoelectric sensor as the sensor 11 facilitates a reduction in thickness. Therefore, it is possible to reduce the possibility of making the subject W1 feel discomfort. In a case where the sensor 11 is a piezoelectric sensor, the sensor 11 is capable of detecting vibrations which have been generated from the subject W1, even when the sensor 11 is disposed at a position at which the sensor 11 does not directly touch the subject W1. This makes it possible to carry out long-term monitoring or constant monitoring of a peristaltic sound without making the subject W1 feel discomfort. Specific examples of the piezoelectric sensor include a piezoelectric sensor in which an electric current occurs in response to compressive deformation, a piezoelectric sensor in which an electric current occurs in response to extensional deformation, and a piezoelectric sensor in which an electric current occurs in response to torsional deformation. In a case where a peristaltic sound, a heart sound, a body motion, and the like are desired to be detected with higher accuracy, it is preferable to apply, as the sensor 11, a piezoelectric sensor in which an electric current occurs upon compressive deformation. In a case where a signal having a low frequency is desired to be detected with higher accuracy, it is preferable to apply, as the sensor 11, a piezoelectric sensor which includes a foam.

A detection signal is a signal (waveform data) itself indicating vibrations which have been generated from the subject W1 or alternatively a signal obtained by subjecting the signal to an amplification process or a noise removal process. Noise processing can be carried out, for example, by filtering a desired frequency region. The sensor 11 can detect vibrations which are generated from the subject W1 and which are in a variety of frequency regions. That is, a detection signal outputted from the sensor 11 is a signal in which a plurality of vibrations having different frequency characteristics overlap each other.

The sensor 11 is preferably capable of detecting vibrations in a wide frequency band. This eliminates the need for disposing a plurality of types of sensors for different frequency bands, makes it easy for the medical expert W2 to carry out maintenance and management, and therefore improves convenience.

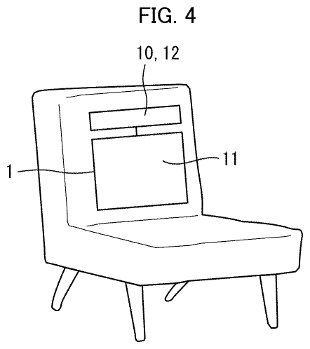

FIGS. 3 to 5 each illustrate an example schematic configuration of the information processing apparatus 1. In order that the subject W1 does not feel discomfort, the sensor 11 is preferably installed at a given position at which the sensor 11 does not touch the subject W1. As illustrated in FIG. 1, the sensor 11 may be installed on a bed on which the subject W1 lies, at a position at which the trunk is supported. In a case where the sensor 11 is installed on a bed, the sensor 11 is preferably formed in the shape of a thin plate (sheet), as illustrated in FIG. 3. Alternatively, the sensor 11 may be installed on a chair on which the subject W1 sits, at a position at which the trunk is supported, as illustrated in FIG. 4. This makes it possible for the sensor 11 to detect vibrations which have been generated from the subject W1 who is lying on a bed or sitting on a chair, i.e., who is in a natural posture.

In a case where the sensor 11 is installed on a bed, the sensor 11 may be installed, as an example, between the bed on which the subject W1 lies and a mattress which is placed on the bed. The sensor 11 may be installed between a bed sheet which is placed on the mattress and the mattress. In a case where the subject W1 wears clothes, the sensor 11 may be installed on the top surface of the bed.

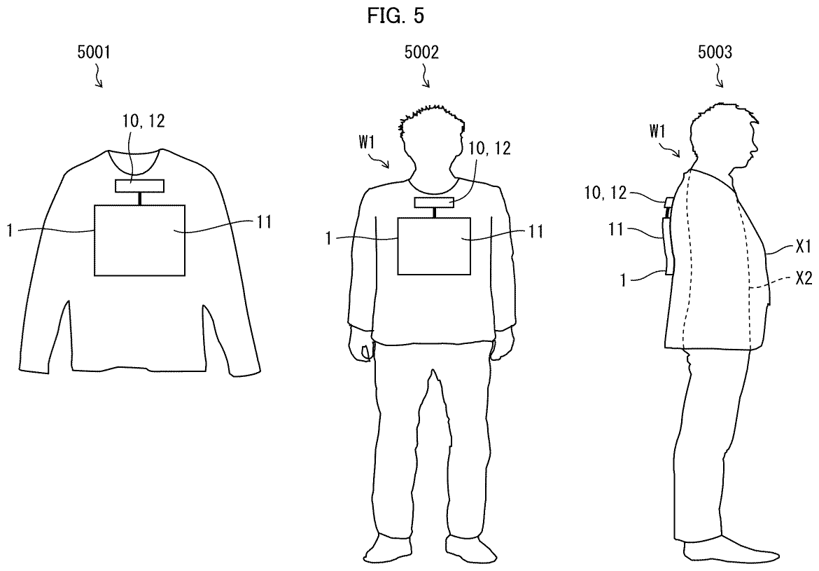

Alternatively, the sensor 11 may be installed on a piece of clothing worn by the subject W1, as illustrated in FIG. 5. 5001 of FIG. 5 illustrates an example installation of the sensor 11 on a piece of clothing. 5002 and 5003 of FIG. 5 each illustrate an example in which the subject W1 is wearing a piece of clothing on which the sensor 11 is installed. As illustrated in 5001 of FIG. 5, the sensor 11 may be installed on a piece of clothing so as to be located on the chest side of the subject W1 when the subject W1 wears the piece of clothing. As illustrated in 5003 of FIG. 5, the sensor 11 may be installed on a piece of clothing so as to be located on the back side of the subject W1 when the subject W1 wears the piece of clothing.

In a case where the sensor 11 of the information processing apparatus 1 is installed on a piece of clothing worn by the subject W1, the sensor 11 may be installed on the piece of clothing (reference sign x₁ in FIG. 5) worn by the subject W1, at a position at which the piece of clothing is sandwiched between the sensor 11 and the trunk (reference sign x₂ in FIG. 5), as illustrated in 5003 of FIG. 5. In this case, it is possible to detect vibrations which have been generated from the subject W1 without touching the sensor 11 to the subject W1. Moreover, with the above configuration, even in a case where the subject W1 has moved from one place to another, it is possible to measure a peristaltic sound in the stomach and intestines of the subject W1, provided that the subject W1 is wearing the piece of clothing which includes the sensor 11. For example, also in a case where the subject W1 who has been sitting on a chair and leaning against a backrest moves to and lies on a bed, it is possible to measure a peristaltic sound in the stomach and intestines of the subject W1.

In a case where the sensor 11 is installed at any of these positions, the sensor 11 is typically preferably formed in the shape of a thin plate (sheet). This makes it possible to detect vibrations which have been generated from the subject W1, without causing discomfort to the subject W1 using a bed or a chair or wearing clothes.

The sensor 11 may include one or more detection areas. In a case where the sensor 11 includes a plurality of detection areas, the sensor 11 may output detection signals which have been detected in the respective plurality of detection areas.

In the case where the sensor 11 is formed in the shape of a thin plate, the plurality of detection areas may be arranged side by side on the same plane. For example, the information processing apparatus 1 illustrated in 3001 of FIG. 3 includes the sensor 11 which includes a single detection area D. The information processing apparatus 1 illustrated in 3002 of FIG. 3 includes the sensor 11 which includes detection areas D1 to D3 arranged in three columns. The information processing apparatus 1 illustrated in 3003 of FIG. 3 includes the sensor 11 which includes detection areas D1a to D3d arranged in four rows and three columns.

For example, in the case of the information processing apparatus 1 illustrated in 3002 of FIG. 3, detection signals which have been detected in the respective detection areas D1 to D3 are individually outputted. Similarly, in the case of the information processing apparatus 1 illustrated in 3003 of FIG. 3, detection signals which have been detected in the respective detection areas D1a to D3d are individually outputted. Each of the detection areas D1a to D3d may be, for example, a 10-cm square.

In a case where the sensor 11 is configured to include a plurality of detection areas, the information processing apparatus 1 is capable of measuring a peristaltic sound with high accuracy by individually analyzing detection signals which have been detected in the respective detection areas and comparing results of analyses with each other.

### [Control Section 12 and Storage Section 13]

As an example, the control section 12 may be a central processing unit (CPU). The control section 12 retrieves a control program which is software stored in the storage section 13, and loads the control program into a memory such as a random access memory (RAM) so as to control each constituent element included in the information processing apparatus 1. Note that, for simplicity of description, the control program is not illustrated in the storage section 13 illustrated in FIG. 7.

As illustrated in FIG. 5, the control section 12 includes the signal extracting section 121, the posture determining section 122, and the output section 123.

The signal extracting section 121 obtains a detection signal outputted from the sensor 11, and extracts, from the detection signal which has been obtained, a heart sound signal indicating a heart sound of the subject W1 and a peristaltic sound signal indicating a peristaltic sound in the stomach and intestines of the subject W1. The signal extracting section 121 extracts the peristaltic sound signal corresponding to a posture which has been determined by the posture determining section 122 (described later).

In a case where the sensor 11 has a plurality of detection areas, the signal extracting section 121 may extract heart sound signals or peristaltic sound signals for the respective plurality of detection areas.

A heart sound signal typically has a frequency characteristic of not more than 200 Hz. A peristaltic sound signal has a frequency characteristic of having a peak in at least one of (i) the range of 150 Hz to 300 Hz and (ii) the range of 500 Hz to 700 Hz. The signal extracting section 121 applies a known technique, such as frequency separation, to the detection signal so as to (i) extract the heart sound signal and the peristaltic sound signal, which respectively have the above-described frequency characteristics, from the detection signal which has been obtained and (ii) further extract the peristaltic sound signal having a signal strength corresponding to the posture of the subject W1 which posture is determined by the posture determining section 122.

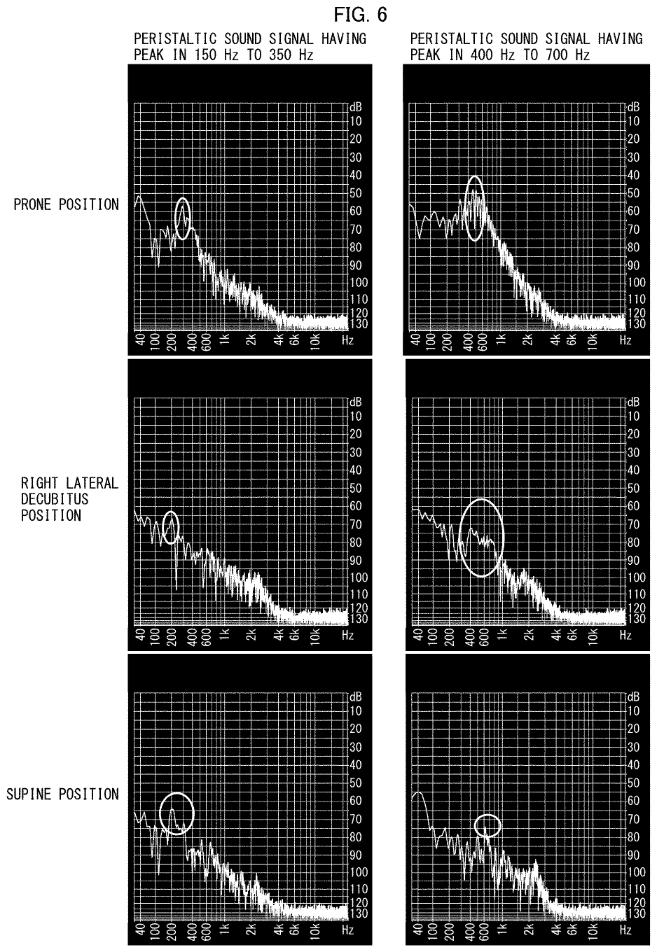

Specific examples of the peristaltic sound signal which is extracted by the signal extracting section 121 and which has a signal strength corresponding to the posture of the subject W1 are illustrated in FIG. 6. FIG. 6 illustrates specific examples of the peristaltic sound signal having a signal strength corresponding to the posture of the subject W1. The upper graphs in FIG. 6 each illustrate a peristaltic sound signal which has been extracted when the sensor is disposed on the abdomen side of the subject W1 lying in a prone position. The middle graphs in FIG. 6 each illustrate a peristaltic sound signal which has been extracted when the sensor is disposed on the right side of the trunk of the subject W1 lying in a right lateral decubitus position. The lower graphs in FIG. 6 each illustrate a peristaltic sound signal which has been extracted when the sensor is disposed on the back side of the subject W1 lying in a supine position. The graphs on the right side each illustrate the peristaltic sound signal having a peak in 150 Hz to 350 Hz, and the graphs on the left side each illustrate the peristaltic sound signal having a peak in 400 Hz to 700 Hz. The circled portion in each of the graphs indicates a peak frequency.

A comparison of signal strengths at these peak frequencies indicates that the peristaltic sound signal having a peak in 150 Hz to 350 Hz or 400 Hz to 700 Hz varies in signal strength depending on the posture.

The signal extracting section 121 may further remove, from the detection signal, the other signals which can be noise. As an example, the other signals which can be noise may be signals corresponding to vibrations generated from machines such as an air conditioner and a television set. From the detection signal, the signal extracting section 121 removes, as noise, for example, signals having the features or characteristics of the signals corresponding to the vibrations generated from the machines such as an air conditioner and a television set. In this case, the features or characteristics of the signals corresponding to the machines only need to be stored in the storage section 13 in advance.

The signal extracting section 121 may store, in the storage section 13, information indicating the detection signal which has been obtained from the sensor 11 and information pertaining to a process carried out by the signal extracting section 121. The information pertaining to the process carried out by the signal extracting section 121 includes information indicating the heart sound signal and the peristaltic sound signal which have been extracted from the detection signal (detection signal 131, heart sound signal 132, and peristaltic sound signal 133 illustrated in FIG. 7). These signals may be stored together with time information indicating the time at which the detection signal, from which these signals have been extracted, was detected.

The posture determining section 122 determines the posture of the subject W1 at the time when the subject W1 generated vibrations. The posture determining section 122 specifies, from a heart sound signal extracted by the signal extracting section 121, a heartbeat-related ballistic motion signal indicating a heartbeat-related ballistic motion of the subject W1, and determines the posture of the subject W1 on the basis of the heartbeat-related ballistic motion signal which has been specified.

When determining the posture of the subject W1, the posture determining section 122 may determine the posture of the subject W1 by inputting, into a posture determination model, the heartbeat-related ballistic motion signal specified from the heart sound signal which has been extracted by the signal extracting section 121. The posture determination model is a model obtained by machine learning with use of training data in which a heartbeat-related ballistic motion signal is an explanatory variable and the posture of the subject W1 which posture corresponds to the heartbeat-related ballistic motion signal is a response variable. A known machine learning algorithm can be applied to the machine learning for generating the posture determination model. Note that the posture determination model may be stored in the storage section 13 or may be stored in an apparatus other than the information processing apparatus 1.

In a case where the sensor 11 has a plurality of detection areas, the posture determining section 122 determines the posture of the subject W1 on the basis of a result of comparison of the frequency characteristics and/or the signal strengths of heartbeat-related ballistic motion signals specified from heart sound signals which have been extracted for the respective detection areas.

In a case where the sensor 11 has the plurality of detection areas, when determining the posture of the subject W1, the posture determining section 122 may determine the posture of the subject W1 by inputting, into a posture determination model, the heartbeat-related ballistic motion signals specified from the heart sound signals which have been extracted by the signal extracting section 121 for the respective plurality of detection areas. The posture determination model is a model obtained by machine learning with use of training data in which heartbeat-related ballistic motion signals, the number of which is the same as the number of the plurality of detection areas, are an explanatory variable and the posture of the subject W1 which posture corresponds to the heartbeat-related ballistic motion signals is a response variable. A known machine learning algorithm can be applied to the machine learning for generating the posture determination model. Note that the posture determination model may be stored in the storage section 13 or may be stored in an apparatus other than the information processing apparatus 1.

The posture determining section 122 may store, in the storage section 13, a result of a process carried out by the posture determining section 122. The result of the process carried out by the posture determining section 122 can be information indicating the heartbeat-related ballistic motion signal specified from the heart sound signal which has been extracted by the signal extracting section 121 (heartbeat-related ballistic motion signal 134 illustrated in FIG. 7) or information indicating a result of determination of the posture of the subject W1 (determination result 136 illustrated in FIG. 7).

The output section 123 outputs, to an external apparatus, a detection signal outputted from the sensor 11, a result of a process carried out in the control section 12, or various pieces of information stored in the storage section 13. As an example, the output section 123 outputs, to the communication apparatus 3, (i) information indicating the detection signal outputted from the sensor 11, (ii) information indicating a heart sound signal and a peristaltic sound signal which have been extracted by the signal extracting section 121, or (iii) information indicating a heartbeat-related ballistic motion signal which has been specified by the posture determining section 122 and information indicating a result of determination of a posture.

A process reference 135 which is information indicating a reference in the above-described process (e.g., extraction of signals, determination of a posture, etc.) that can be carried out by each section included in the control section 12 may be stored in the storage section 13.

### (Configuration of Communication Apparatus 3)

A configuration of the communication apparatus 3 is described with reference to FIG. 7. The communication apparatus 3 communicates with the information processing apparatus 1 via the communication network 9, and receives information outputted from the information processing apparatus 1. The communication apparatus 3 may transmit, to the information processing apparatus 1, a request for transmission of various pieces of information, as necessary.

As illustrated in FIG. 5, the communication apparatus 3 includes an input section 31, a control section 32, a storage section 33, and a display section 34.

The input section 31 accepts an input operation conducted by a user with respect to the communication apparatus 3, and transmits, to the control section 32, a signal (instruction signal) corresponding to the input operation. The input operation conducted by the user is, for example, switching of subject information displayed by the display section 34. Specific examples of the input section 31 include a keyboard, a touch panel, and a mouse.

The control section 32 controls each constituent element included in the communication apparatus 3. As an example, the control section 32 retrieves a control program (not illustrated) which is software stored in the storage section 33, and loads the control program into a memory such as a RAM so as to control each constituent element included in the communication apparatus 3. The control section 32 includes an input accepting section 321 which obtains an instruction signal transmitted by the input section 31 and a display control section 322 which outputs various pieces of information to the display section 34.

In the storage section 33, various pieces of information are stored. As an example, in addition to the above-described control program, (i) information indicating a detection signal which has been received by the signal extracting section 121 from the sensor 11, (ii) information indicating a heart sound signal and a peristaltic sound signal which have been extracted by the signal extracting section 121, or (iii) information indicating a heartbeat-related ballistic motion signal which has been specified by the posture determining section 122 and information indicating a result of determination of a posture is stored in the storage section 33.

The display section 34 displays a display image which has been outputted from the control section 32. As an example, the display section 34 displays the display image including at least one of (i) information indicating a detection signal which is received from the information processing apparatus 1 and which has been received by the signal extracting section 121, (ii) information indicating a heart sound signal and a peristaltic sound signal which have been extracted by the signal extracting section 121, and (iii) information indicating a heartbeat-related ballistic motion signal which has been specified by the posture determining section 122 and information indicating a result of determination of a posture.

### (Processes Carried Out By Information Processing System 100)

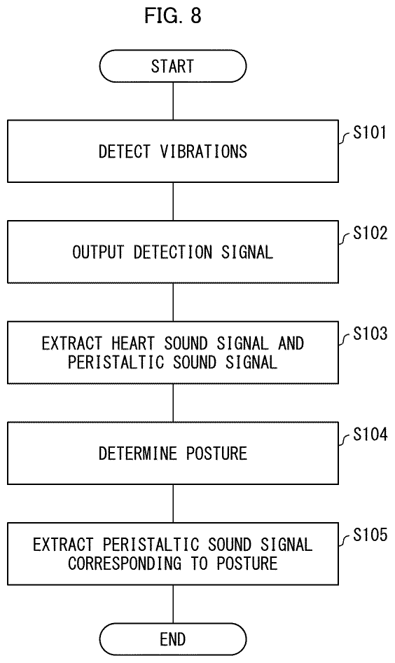

Next, a flow of processes carried out by the information processing system 100 is described with reference to FIG. 8. FIG. 8 is a flowchart illustrating an example flow of the processes carried out by the information processing system 100 (for example, information processing apparatus 1).

First, the sensor 11 that is included in the information processing apparatus 1 and that is installed at a given position at which the sensor 11 does not touch a subject W1 detects vibrations which have been generated from the subject W1 (S101). The sensor 11 outputs, to the control section 12, a detection signal corresponding to the vibrations which have been detected (S102: outputting step).

In a case where the signal extracting section 121 of the control section 12 obtains the detection signal from the sensor 11, the signal extracting section 121 extracts a heart sound signal and a peristaltic sound signal from the detection signal (S103: first extracting step). The signal extracting section 121 outputs, to the posture determining section 122, the heart sound signal which has been extracted.

The posture determining section 122 determines the posture of the subject W1 on the basis of a heartbeat-related ballistic motion signal specified from the heart sound signal which has been obtained from the signal extracting section 121 (S104: determining step). The posture determining section 122 outputs a result of determination of the posture to the signal extracting section 121.

The signal extracting section 121 extracts the peristaltic sound signal corresponding to the posture which has been determined in S104, on the basis of (i) the result of the determination of the posture which result has been obtained from the posture determining section 122 and (ii) the peristaltic sound signal which has been extracted in the step S103 (S105: second extracting step).

After extraction of the peristaltic sound signal corresponding to the posture, the output section 123 may output, to the communication apparatus 3, various pieces of information including information indicating the peristaltic sound signal, and the display control section 322 may control the display section 34 to display the various pieces of information which have been obtained.

Note that the timing when the information processing system 100 carries out the processes illustrated in FIG. 8 can be set as desired. For example, the information processing system 100 may carry out the processes illustrated in FIG. 8 every given period of time (for example, 1 hour), or may carry out the processes illustrated in FIG. 8 every time it is determined via the sensor 11 that the subject W1 has got out of a bed.

### (Variation 1 of Configuration of Information Processing System 100)

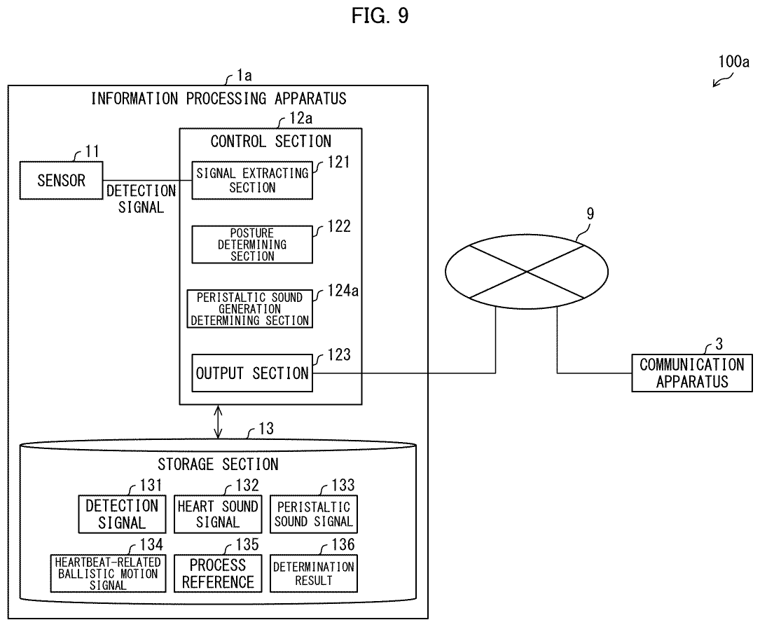

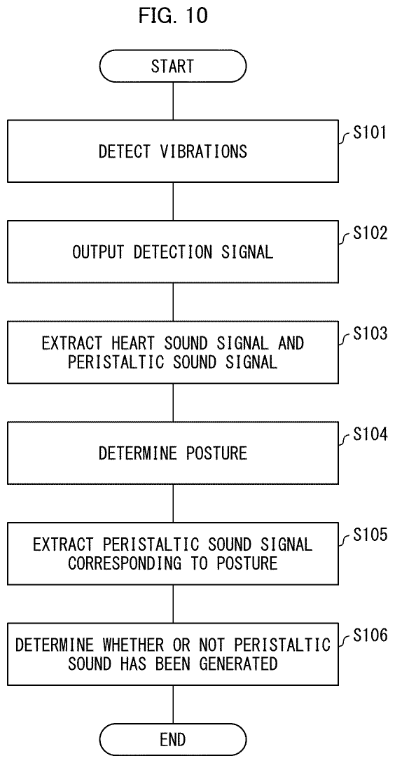

Variation 1 (hereinafter, referred to as “information processing system 100a”) of the configuration of the information processing system 100 is described with reference to FIGS. 9 and 10. For convenience, members having the same functions as those of the above-described members are given the same reference signs, and descriptions thereof are not repeated.

[Configuration of Information Processing System 100a]

A configuration of the information processing system 100a is described with reference to FIG. 9. FIG. 9 is a functional block diagram illustrating an example configuration of the information processing system 100a.

As illustrated in FIG. 9, the information processing system 100a differs from the information processing system 100 in that the information processing system 100a includes an information processing apparatus 1a instead of the information processing apparatus 1. In the other configurations, the information processing system 100a is the same as the information processing system 100.

As illustrated in FIG. 9, the information processing apparatus 1a differs from the information processing apparatus 1 in that the information processing apparatus la includes a control section 12a instead of the control section 12. The control section 12a differs from the control section 12 in that the control section 12a further includes a peristaltic sound generation determining section 124a.

The peristaltic sound generation determining section 124a determines whether or not a peristaltic sound has been generated in vibrations generated from a subject W1. As an example, the peristaltic sound generation determining section 124a determines that a peristaltic sound has been generated, in a case where a peristaltic sound signal extracted by a signal extracting section 121 has at least one of (i) a given frequency characteristic and (ii) a given signal strength at a given frequency.

An example of the “given frequency characteristic” is a frequency characteristic of having a peak in at least one of (i) the range of 150 Hz to 300 Hz and (ii) the range of 500 Hz to 700 Hz, which is the frequency characteristic of a peristaltic sound.

An example of the “given signal strength at a given frequency” may be a signal strength at or around 200 Hz or 600 Hz in a state in which a peristaltic sound having a peak in at least one of (i) the range of 150 Hz to 300 Hz and (ii) the range of 500 Hz to 700 Hz has been obtained.

[Processes Carried Out By Information Processing System 100a]

A flow of processes carried out by the information processing system 100a is described with reference to FIG. 10. FIG. 10 is a flowchart illustrating an example flow of the processes carried out by the information processing system 100a (for example, information processing apparatus 1a).

As illustrated in FIG. 10, the information processing system 100a carries out processes in steps S101 to S106. S101 to S105 are similar to the steps illustrated in FIG. 8, and therefore descriptions thereof are omitted here.

In a case where the peristaltic sound generation determining section 124a obtains a peristaltic sound signal which has been extracted by the signal extracting section 121 in S105 and which corresponds to the posture of a subject W1, the peristaltic sound generation determining section 124a determines whether or not a peristaltic sound has been generated in the stomach and intestines of the subject W1, on the basis of the peristaltic sound signal (S106). The peristaltic sound generation determining section 124a determines that a peristaltic sound has been generated, in a case where the peristaltic sound signal which has been extracted by the signal extracting section 121 and which corresponds to the posture of the subject W1 has at least one of (i) a given frequency characteristic and (ii) a given signal strength at a given frequency. The peristaltic sound generation determining section 124a may output a result of determination to an output section 123, and the output section 123 may output, to a communication apparatus 3, information including the result of the determination.

Note that the timing when the information processing system 100a carries out the processes illustrated in FIG. 10 can be set as desired. For example, the information processing system 100a may carry out the processes illustrated in FIG. 10 every given period of time (for example, 1 hour), or may carry out the processes illustrated in FIG. 10 every time it is determined via a sensor 11 that the subject W1 has got out of a bed.

### (Variation 2 of Configuration of Information Processing System 100)

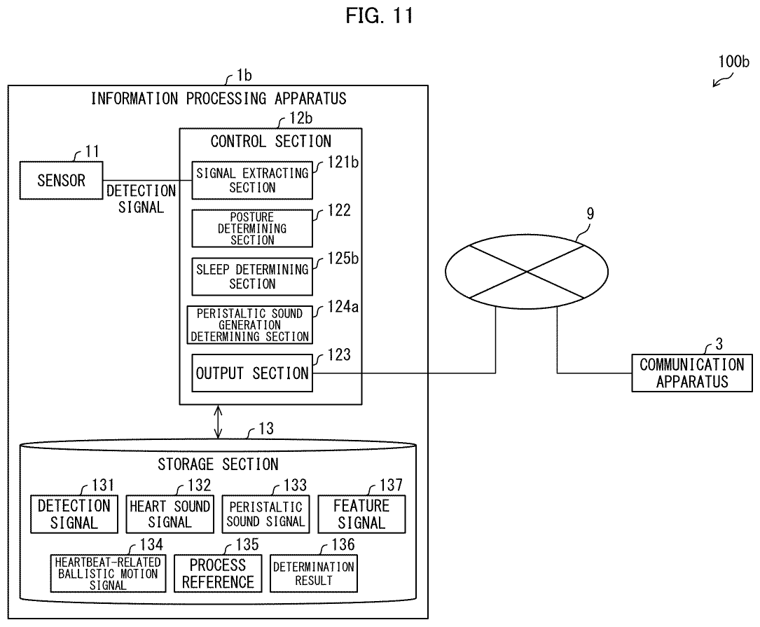

Variation 2 (hereinafter, referred to as “information processing system 100b”) of the configuration of the information processing system 100 is described with reference to FIGS. 11 to 13. For convenience, members having the same functions as those of the above-described members are given the same reference signs, and descriptions thereof are not repeated.

[Configuration of Information Processing System 100b]

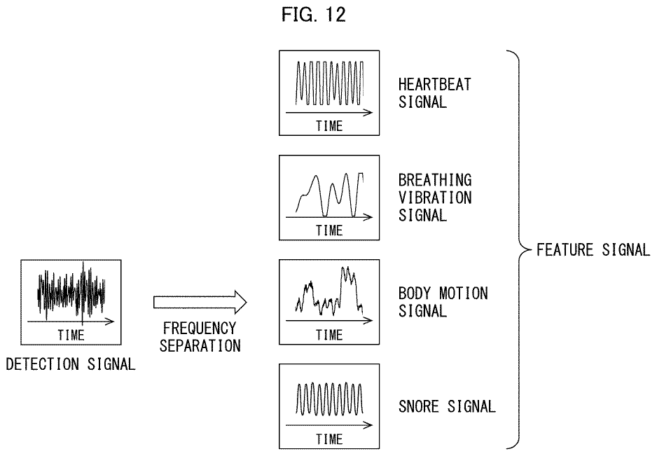

A configuration of the information processing system 100b is described with reference to FIGS. 11 and 12. FIG. 11 is a functional block diagram illustrating an example configuration of the information processing system 100b. FIG. 12 illustrates a feature signal included in a detection signal.

As illustrated in FIG. 11, the information processing system 100b differs from the information processing system 100a in that the information processing system 100b includes an information processing apparatus 1b instead of the information processing apparatus 1a. In the other configurations, the information processing system 100b is the same as the information processing system 100a.

As illustrated in FIG. 11, the information processing apparatus 1b differs from the information processing apparatus 1a in that the information processing apparatus 1b includes a control section 12b instead of the control section 12a. The control section 12b differs from the control section 12a in that the control section 12b includes a signal extracting section 121b instead of the signal extracting section 121 and further includes a sleep determining section 125b.

The signal extracting section 121b differs from the signal extracting section 121 in that the signal extracting section 121b further extracts a feature signal from a detection signal outputted from a sensor 11. Note, here, that, as illustrated in FIG. 12, a “feature signal” includes at least one of (i) a heartbeat signal which indicates a heartbeat of a subject W1, (ii) a breathing vibration signal which indicates a breathing vibration of the subject W1, (iii) a body motion signal which indicates a body motion of the subject W1, and (iv) a snore signal which indicates a snore of the subject W1.

The sleep determining section 125b determines the sleep state of the subject W1. As an example, the sleep determining section 125b determines the sleep state of the subject W1 on the basis of the feature signal which has been extracted by the signal extracting section 121b.

When determining the sleep state of the subject W1, the sleep determining section 125b may determine the sleep state of the subject W1 as follows. That is, into a sleep determination model obtained by machine learning with use of training data in which a set constituted by, as an element(s), at least one of the above-listed signals that is included in a feature signal is an explanatory variable and the sleep state of the subject W1 which sleep state corresponds to the set is a response variable, the sleep determining section 125b inputs a set constituted by an element(s) which is/are included in the feature signal that has been extracted by the signal extracting section 121b and which is/are identical to the element(s) of the explanatory variable, thereby determining the sleep state of the subject W1. A known machine learning algorithm can be applied to the machine learning for generating the sleep determination model. Note that the sleep determination model may be stored in a storage section 13 or may be stored in an apparatus other than the information processing apparatus 1b.

[processes Carried Out By Information Processing System 100b]

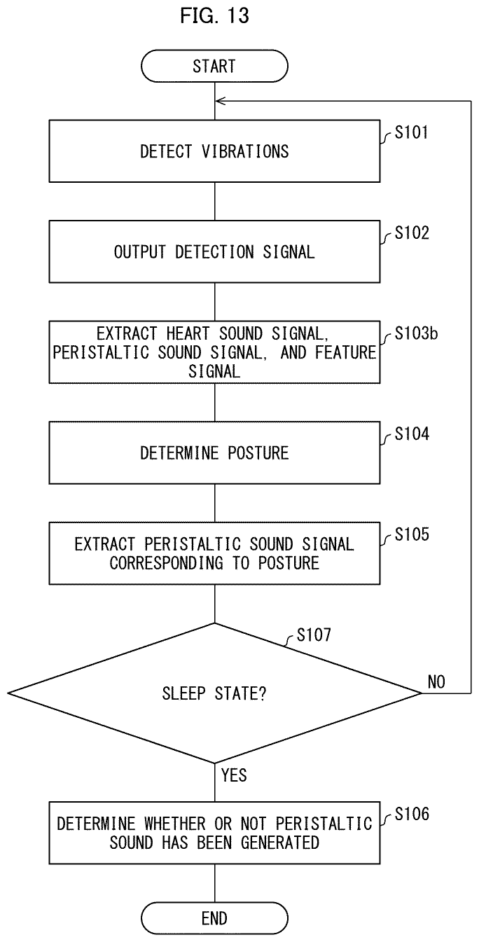

A flow of processes carried out by the information processing system 100b is described with reference to FIG. 13. FIG. 13 is a flowchart illustrating an example flow of the processes carried out by the information processing system 100b.

As illustrated in FIG. 13, the information processing system 100b carries out processes in steps S101, S102, S103b, S104, S105, S107, and S106. Descriptions of S101, S102, S104, S105, and S106 are similar to the descriptions given above, and are therefore omitted.

The signal extracting section 121b extracts a heart sound signal, a peristaltic sound signal, and a feature signal from a detection signal which has been obtained (S103b). The signal extracting section 121b outputs, to a posture determining section 122, the heart sound signal which has been extracted, and outputs, to the sleep determining section 125b, the detection signal and the feature signal which has been extracted.

The sleep determining section 125b determines the sleep state of a subject W1 on the basis of at least one of the detection signal and the feature signal which have been obtained from the signal extracting section 121b (S107). Specifically, the sleep determining section 125b determines whether or not the subject W1 is in the sleep state.

In the case of YES in the step S107, that is, in a case where the sleep determining section 125b determines that the subject W1 is in the sleep state, the peristaltic sound generation determining section 124a carries out a process in the step S106. In the case of NO in the step S107, that is, in a case where the sleep determining section 125b determines that the subject W1 is not in the sleep state, the flow returns to the step S101.

### Embodiment 2

The following description will discuss Embodiment 2 of the present disclosure. Note that, in embodiments below, members having the same functions as those of the members described in the above embodiment are given the same reference signs, and descriptions thereof are not repeated for convenience.

### (Configuration of Information Processing System 100c)

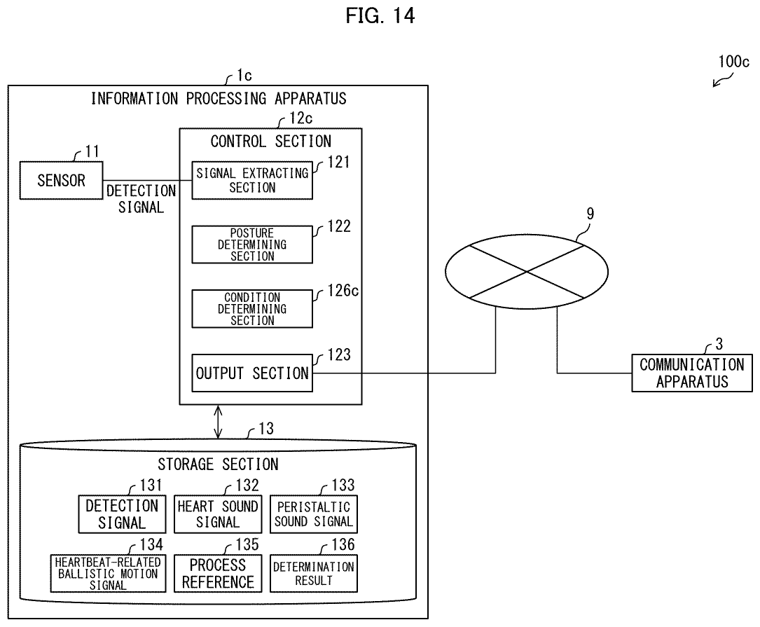

FIG. 14 is a functional block diagram illustrating an example configuration of an information processing system 100c in accordance with Embodiment 2. As illustrated in FIG. 14, the information processing system 100c differs from the information processing system 100 in that the information processing system 100c includes an information processing apparatus lc instead of the information processing apparatus 1. In the other configurations, the information processing system 100c is the same as the information processing system 100.

As illustrated in FIG. 14, the information processing apparatus 1c differs from the information processing apparatus 1 in that the information processing apparatus 1c includes a control section 12c instead of the control section 12. The control section 12c differs from the control section 12 in that the control section 12c includes a condition determining section 126c.

The condition determining section 126c determines the condition of the stomach and intestines of a subject W1 on the basis of a peristaltic sound signal extracted by a signal extracting section 121. As an example, in a case where the peristaltic sound signal extracted by the signal extracting section 121 has a peak at or around 600 Hz and the peristaltic sound signal is extracted 12 or more times per minute, the condition determining section 126c determines that the condition of the stomach and intestines of the subject W1 is diarrhea or ileus. In a case where the peristaltic sound signal extracted by the signal extracting section 121 has a peak at or around 200 Hz and the peristaltic sound signal is extracted 1 to 3 times per minute, the condition determining section 126c determines that the condition of the stomach and intestines of the subject W1 is constipation. In a case where no peristaltic sound signal can be extracted for 5 or more minutes, the condition determining section 126c determines that the condition of the stomach and intestines of the subject W1 is ileus or peritonitis.

When determining the condition of the stomach and intestines of the subject W1, the condition determining section 126c may determine the condition of the stomach and intestines of the subject W1 by inputting, into a condition determination model, the peristaltic sound signal which has been extracted by the signal extracting section 121. The condition determination model is a model obtained by machine learning with use of training data in which a peristaltic sound signal that has been extracted by the signal extracting section 121 is an explanatory variable and the condition of the stomach and intestines of the subject W1 which condition corresponds to the peristaltic sound signal that has been extracted is a response variable. A known machine learning algorithm can be applied to the machine learning for generating the condition determination model. Note that the condition determination model may be stored in a storage section 13 or may be stored in an apparatus other than the information processing apparatus 1c.

### (Processes Carried Out By Information Processing System 100c)

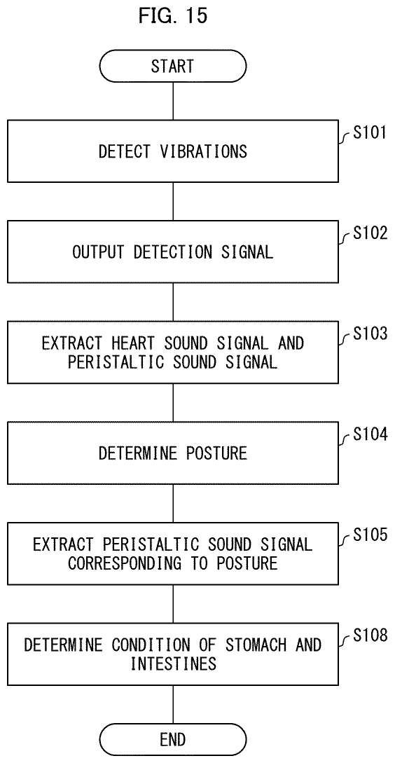

A flow of processes carried out by the information processing system 100c is described with reference to FIG. 15. FIG. 15 is a flowchart illustrating an example flow of the processes carried out by the information processing system 100c.

As illustrated in FIG. 15, the information processing system 100c carries out processes in steps S101 to S105 and S108. Descriptions of the steps S101 to S105 are similar to the descriptions given above, and are therefore omitted.

The condition determining section 126c obtains a peristaltic sound signal which has been extracted by the signal extracting section 121 and which corresponds to the posture of a subject W1, and determines the condition of the stomach and intestines of the subject W1 on the basis of the peristaltic sound signal (S108). The condition determining section 126c may output result of determination to an output section 123, and the output section 123 may output, to a communication apparatus 3, information including the result of the determination.

### Embodiment 3

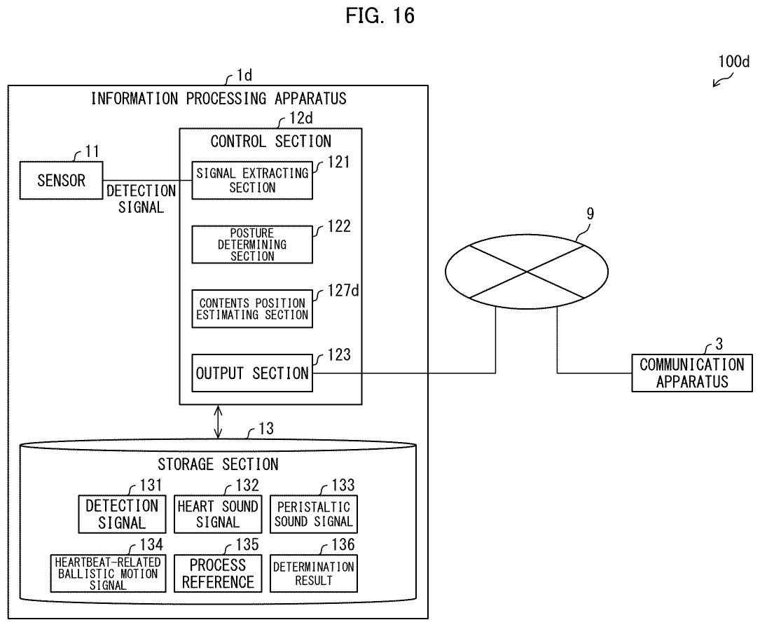

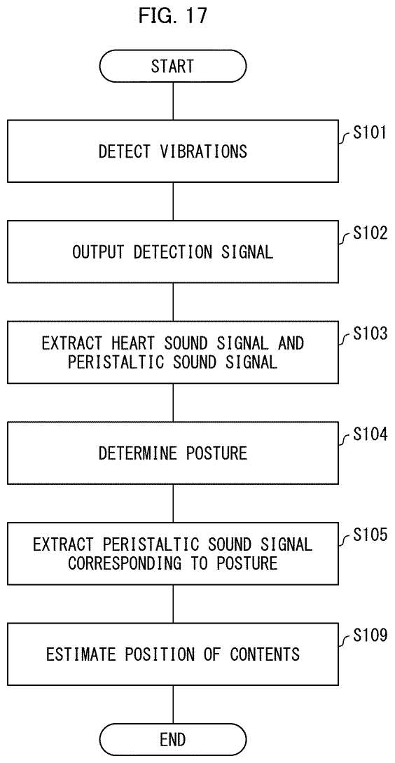

The following description will discuss Embodiment 3 of the present disclosure with reference to FIGS. 16 and 17. For convenience, members having the same functions as those of the members described in the above embodiments are given the same reference signs, and descriptions thereof are not repeated.

### (Configuration of Information Processing System 100d)

An information processing system 100d which is configured such that a sensor 11 includes a plurality of detection areas and outputs detection signals which have been detected in the respective plurality of detection areas is described with reference to FIG. 16.

FIG. 16 is a functional block diagram illustrating an example configuration of the information processing system 100d in accordance with Embodiment 3. As illustrated in FIG. 16, the information processing system 100d differs from the information processing system 100 in that the information processing system 100d includes an information processing apparatus 1d instead of the information processing apparatus 1. In the other configurations, the information processing system 100d is the same as the information processing system 100.

As illustrated in FIG. 14, the information processing apparatus 1d differs from the information processing apparatus 1 in that the information processing apparatus 1d includes a control section 12d instead of the control section 12. The control section 12d differs from the control section 12 in that the control section 12d further includes a contents position estimating section 127d.

The contents position estimating section 127d estimates the position of contents in the stomach and intestines of a subject W1. The contents in the stomach and intestines include ingested foods, gases, and stools. As an example, the contents position estimating section 127d estimates the position of the contents in the stomach and intestines of the subject W1 on the basis of peristaltic sound signals which have been extracted by a signal extracting section 121 for the respective plurality of detection areas, arrangement information which indicates arrangement of the plurality of detection areas, and a posture which has been determined by a posture determining section 122.

The contents position estimating section 127d may estimate the position of the contents in the stomach and intestines of the subject W1 by inputting, into a position estimation model, the peristaltic sound signals which have been extracted by the signal extracting section 121 for the respective plurality of detection areas, the arrangement information which indicates the arrangement of the plurality of detection areas, and the posture which has been determined by the posture determining section 122. The position estimation model is a model obtained by machine learning with use of training data in which peristaltic sound signals which have been extracted by the signal extracting section 121 for the respective plurality of detection areas, the arrangement information which indicates the arrangement of the plurality of detection areas, and the posture which has been determined by the posture determining section 122 are an explanatory variable and the position of the contents is a response variable.

### (Processes Carried Out By Information Processing System 100d)

A flow of processes carried out by the information processing system 100d is described with reference to FIG. 17. FIG. 17 is a flowchart illustrating an example flow of the processes carried out by the information processing system 100d.

As illustrated in FIG. 17, the information processing system 100d carries out processes in steps S101 to S105 and S109. Descriptions of the steps S101 to S105 are similar to the descriptions given above, and are therefore omitted.

The contents position estimating section 127d estimates the position of contents in the stomach and intestines of a subject W1 on the basis of peristaltic sound signals which have been extracted by the signal extracting section 121 and which have been obtained, the arrangement information which indicates the arrangement of the plurality of detection areas, and a posture which has been determined by the posture determining section 122 (S109). The contents position estimating section 127d may output a result of estimation to an output section 123, and the output section 123 may output, to a communication apparatus 3, information including the result of the estimation.

### Embodiment 4

An information processing system in accordance with the present invention may be configured such that a peristaltic sound signal indicating a peristaltic sound in a subject W1 is extracted from vibrations which have been generated from the subject W1, as in the information processing system 100 in accordance with Embodiment 1. Alternatively, an information processing system in accordance with the present invention may be configured such that a peristaltic sound signal indicating a peristaltic sound in the stomach and intestines of a subject W1 is extracted from vibrations which have been generated from the subject W1 and the position of generation of the peristaltic sound is estimated, as in an information processing system 200 in accordance with Embodiment 4.

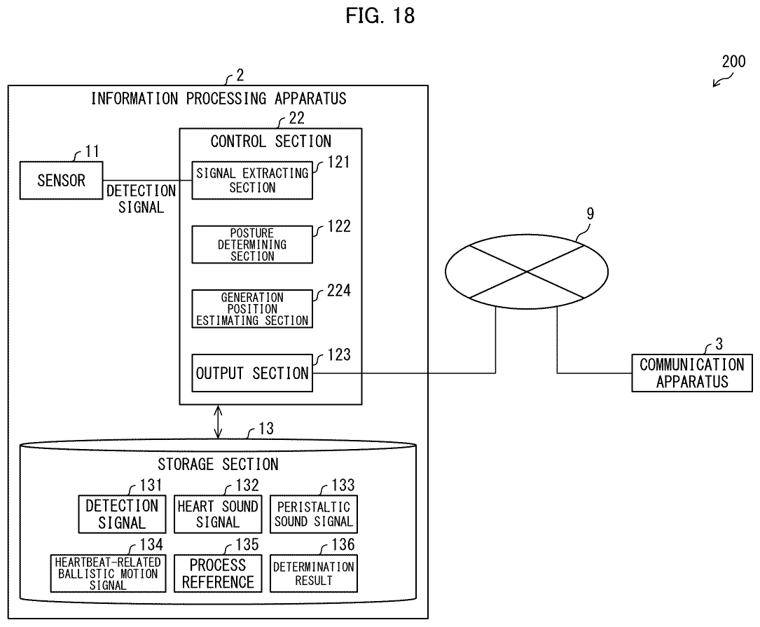

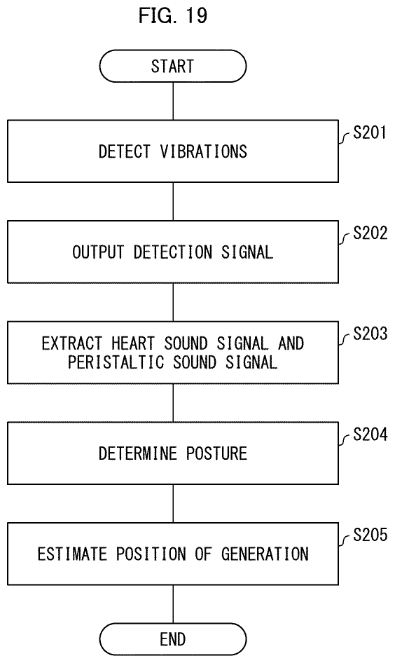

The following description will discuss Embodiment 4 of the present disclosure with reference to FIGS. 18 and 19. For convenience, members having the same functions as those of the members described in the above embodiments are given the same reference signs, and descriptions thereof are not repeated.

### (Outline of Information Processing System 200)

The information processing system 200 in accordance with Embodiment 4 of the present disclosure is a system in which (i) a peristaltic sound signal indicating a peristaltic sound in a subject W1 is extracted on the basis of a detection signal outputted from a sensor that detects, at a position at which the sensor does not touch the subject W1, vibrations which have been generated from the subject W1 and (ii) the position of generation of the peristaltic sound signal is estimated.

### (Configuration of Information Processing System 200)

FIG. 18 is a functional block diagram illustrating an example configuration of the information processing system 200 in accordance with Embodiment 4. As illustrated in FIG. 18, the information processing system 200 differs from the information processing system 100 in that the information processing system 200 includes an information processing apparatus 2 instead of the information processing apparatus 1. In the other configurations, the information processing system 200 is the same as the information processing system 100.

As illustrated in FIG. 18, the information processing apparatus 2 differs from the information processing apparatus 1 in that the information processing apparatus 2 includes a control section 22 instead of the control section 12. The control section 22 differs from the control section 12 in that the control section 22 further includes a generation position estimating section 224.

The generation position estimating section 224 estimates the position of generation of a peristaltic sound indicated by a peristaltic sound signal which has been extracted by a signal extracting section 121, on the basis of the peristaltic sound signal and a posture which has been determined by a posture determining section 122.

### (Processes Carried Out By Information Processing System 200)

A flow of processes carried out by the information processing system 200 is described with reference to FIG. 19. FIG. 19 is a flowchart illustrating an example flow of the processes carried out by the information processing system 200.

First, a sensor 11 that is included in the information processing apparatus 2 and that is installed at a given position at which the sensor 11 does not touch a subject W1 detects vibrations which have been generated from the subject W1 (S201). The sensor 11 outputs, to the control section 22, a detection signal corresponding to the vibrations which have been detected (S202: outputting step).

In a case where the signal extracting section 121 of the control section 22 obtains the detection signal from the sensor 11, the signal extracting section 121 extracts a heart sound signal and a peristaltic sound signal from the detection signal (S203: extracting step). The signal extracting section 121 outputs, to the posture determining section 122, the heart sound signal which has been extracted, and outputs, to the generation position estimating section 224, the peristaltic sound signal which has been extracted.

The posture determining section 122 determines the posture of the subject W1 on the basis of a heartbeat-related ballistic motion signal specified from the heart sound signal which has been obtained from the signal extracting section 121 (S204: determining step). The posture determining section 122 outputs a result of determination of the posture to the generation position estimating section 224.

The generation position estimating section 224 estimates the position of generation of a peristaltic sound indicated by the peristaltic sound, on the basis of (i) the result of the determination which result has been obtained from the posture determining section 122 and (ii) the peristaltic sound signal which has been obtained from the signal extracting section 121 (S205: estimating step). The generation position estimating section 224 may output a result of estimation to an output section 123, and the output section 123 may output, to a communication apparatus 3, information including the result of the estimation.

### (Variation of Configuration of Information Processing System 200)

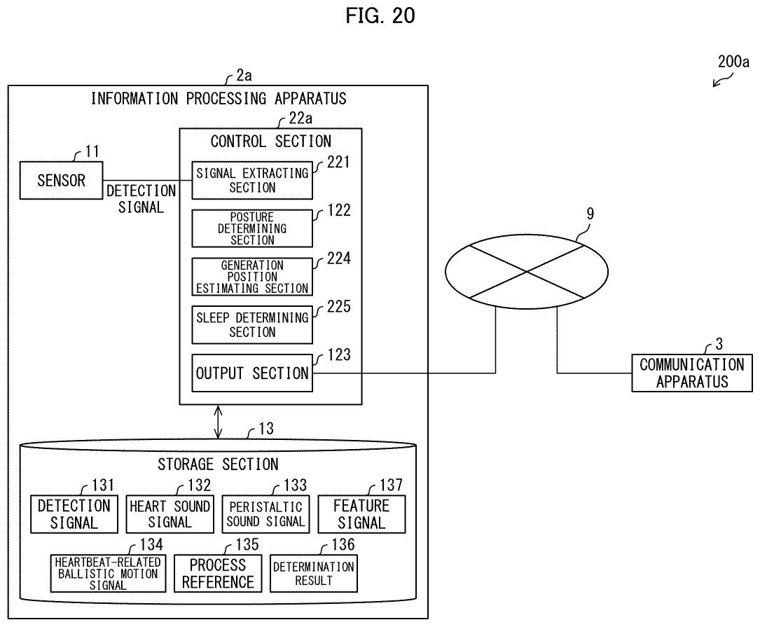

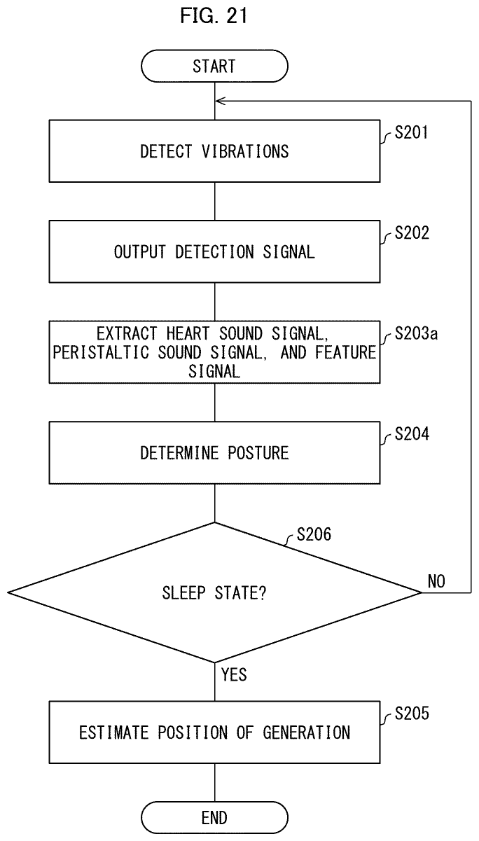

A variation (hereinafter, referred to as “information processing system 200a”) of the configuration of the information processing system 200 is described with reference to FIGS. 20 and 21. For convenience, members having the same functions as those of the above-described members are given the same reference signs, and descriptions thereof are not repeated.

[Configuration of Information Processing System 200a]

A configuration of the information processing system 200a is described with reference to FIG. 20. FIG. 20 is a functional block diagram illustrating an example configuration of the information processing system 200a.

As illustrated in FIG. 20, the information processing system 200a differs from the information processing system 200 in that the information processing system 200a includes an information processing apparatus 2a instead of the information processing apparatus 2. In the other configurations, the information processing system 200a is the same as the information processing system 200.

As illustrated in FIG. 20, the information processing apparatus 2a differs from the information processing apparatus 2 in that the information processing apparatus 2a includes a control section 22a instead of the control section 22. The control section 22a differs from the control section 22 in that the control section 22a includes a signal extracting section 221 instead of the signal extracting section 121 and further includes a sleep determining section 225.

The signal extracting section 221 is similar to the above-described signal extracting section 121b, and the sleep determining section 225 is similar to the above-described sleep determining section 125b. Thus, descriptions thereof are omitted here.

[Processes Carried Out By Information Processing System 200a]

A flow of processes carried out by the information processing system 200a is described with reference to FIG. 21. FIG. 21 is a flowchart illustrating an example flow of the processes carried out by the information processing system 200a.

As illustrated in FIG. 21, the information processing system 200a carries out processes in steps S201, S202, S203a, S204, S206, and S205. Descriptions of S201, S202, S204, and S205 are similar to the descriptions given above, and are therefore omitted.

The signal extracting section 221 extracts a heart sound signal, a peristaltic sound signal, and a feature signal from a detection signal which has been obtained (S203a). The signal extracting section 221 outputs, to a posture determining section 122, the heart sound signal which has been extracted, and outputs, to the sleep determining section 225, the detection signal and the feature signal which has been extracted.

The sleep determining section 225 determines the sleep state of a subject W1 on the basis of at least one of the detection signal and the feature signal which have been obtained from the signal extracting section 221 (S206). Specifically, the sleep determining section 225 determines whether or not the subject W1 is in the sleep state.

In the case of YES in the step S206, that is, in a case where the sleep determining section 225 determines that the subject W1 is in the sleep state, the generation position estimating section 224 carries out the process in the step S205. In the case of NO in the step S206, that is, in a case where the sleep determining section 225 determines that the subject W1 is not in the sleep state, the flow returns to the step S201.

### Embodiment 5

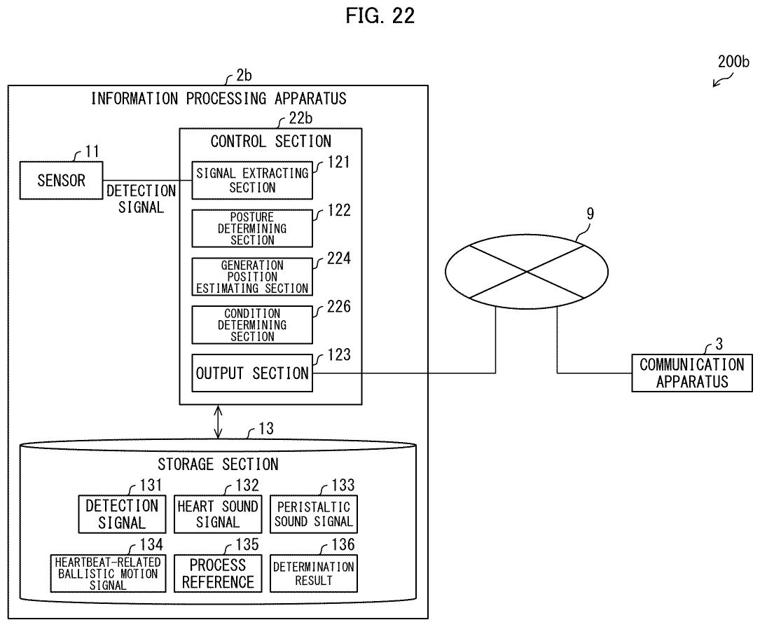

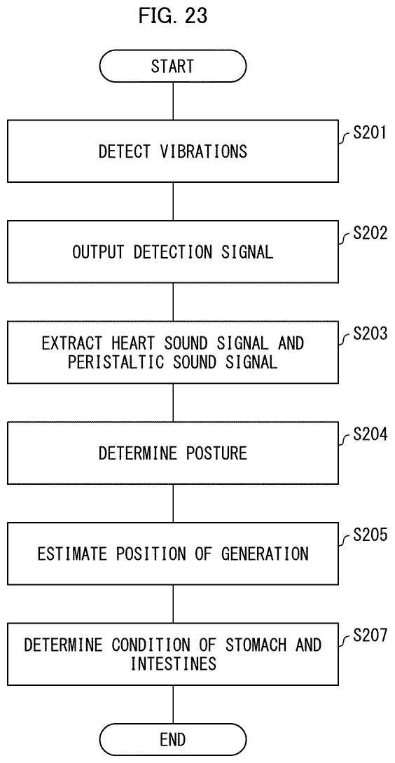

The following description will discuss Embodiment 5 of the present disclosure with reference to FIGS. 22 and 23. For convenience, members having the same functions as those of the members described in the above embodiments are given the same reference signs, and descriptions thereof are not repeated.

### (Configuration of Information Processing System 200b)

FIG. 22 is a functional block diagram illustrating an example configuration of the information processing system 200b in accordance with Embodiment 5. As illustrated in FIG. 22, the information processing system 200b differs from the information processing system 200 in that the information processing system 200b includes an information processing apparatus 2b instead of the information processing apparatus 2. In the other configurations, the information processing system 200b is the same as the information processing system 200.

As illustrated in FIG. 22, the information processing apparatus 2b differs from the information processing apparatus 2 in that the information processing apparatus 2b includes a control section 22b instead of the control section 22. The control section 22b differs from the control section 22 in that the control section 22b includes a condition determining section 226.

The condition determining section 226 is similar to the above-described condition determining section 126c. Thus, a description thereof is omitted here.

### (Processes Carried Out By Information Processing System 200b)

A flow of processes carried out by the information processing system 200b is described with reference to FIG. 23. FIG. 23 is a flowchart illustrating an example flow of the processes carried out by the information processing system 200b.

As illustrated in FIG. 23, the information processing system 200b carries out processes in steps S201 to S205 and S207. Descriptions of the steps S201 to S205 are similar to the descriptions given above, and are therefore omitted.

The condition determining section 226 determines the condition of the stomach and intestines of a subject W1 on the basis of a peristaltic sound signal which has been extracted by a signal extracting section 121 (S207). The condition determining section 226 may output a result of determination to an output section 123, and the output section 123 may output, to a communication apparatus 3, information including the result of the determination.

### Embodiment 6

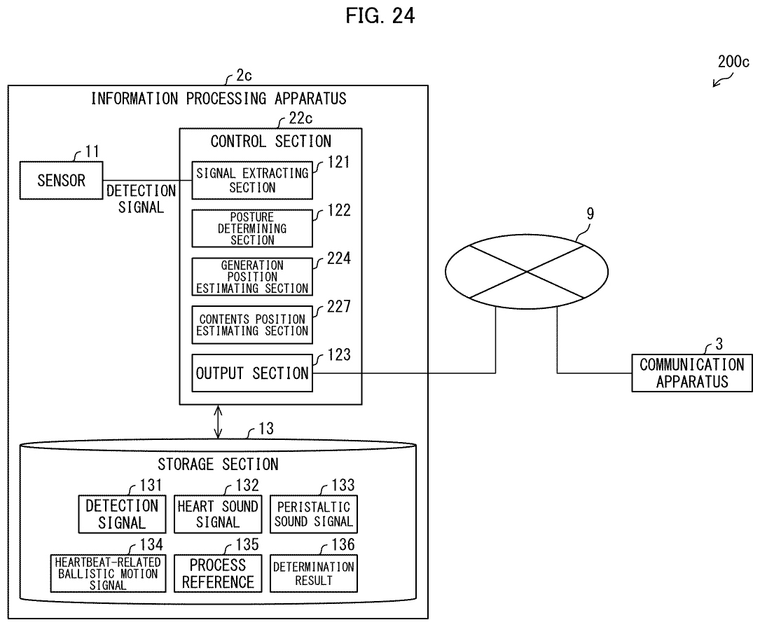

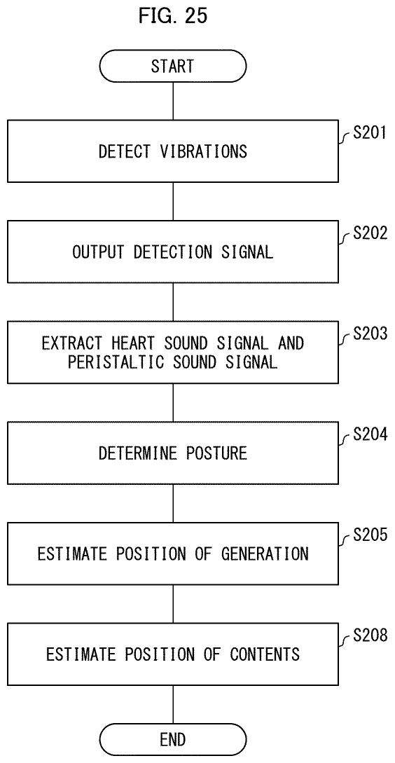

The following description will discuss another embodiment of the present disclosure with reference to FIGS. 24 and 25. For convenience, members having the same functions as those of the members described in the above embodiments are given the same reference signs, and descriptions thereof are not repeated.

### (Configuration of Information Processing System 200c)

An information processing system 200c which is configured such that a sensor 11 includes a plurality of detection areas and outputs detection signals detected in the respective plurality of detection areas is described with reference to FIG. 24.

FIG. 24 is a functional block diagram illustrating an example configuration of the information processing system 200c in accordance with Embodiment 6. As illustrated in FIG. 24, the information processing system 200c differs from the information processing system 200 in that the information processing system 200c includes an information processing apparatus 2c instead of the information processing apparatus 2. In the other configurations, the information processing system 200c is the same as the information processing system 200.

As illustrated in FIG. 24, the information processing apparatus 2c differs from the information processing apparatus 2 in that the information processing apparatus 2c includes a control section 22c instead of the control section 22. The control section 22c differs from the control section 22 in that the control section 22c further includes a contents position estimating section 227.

The contents position estimating section 227 is similar to the above-described contents position estimating section 127d. Thus, a description thereof is omitted here.

### (Processes Carried Out By Information Processing System 200c)

A flow of processes carried out by the information processing system 200c is described with reference to FIG. 25. FIG. 25 is a flowchart illustrating an example flow of the processes carried out by the information processing system 200c.

As illustrated in FIG. 25, the information processing system 200c carries out processes in steps S201 to S205 and S208. Descriptions of the steps S201 to S205 are similar to the descriptions given above, and are therefore omitted.

The contents position estimating section 227 estimates the position of contents in the stomach and intestines of a subject W1, on the basis of peristaltic sound signals which have been extracted by a signal extracting section 121 and which have been obtained, arrangement information which indicates arrangement of the plurality of detection areas, and a posture which has been determined by a posture determining section 122 (S208). The contents position estimating section 227 may output a result of estimation to an output section 123, and the output section 123 may output, to a communication apparatus 3, information including the result of the estimation.

### Embodiment 7

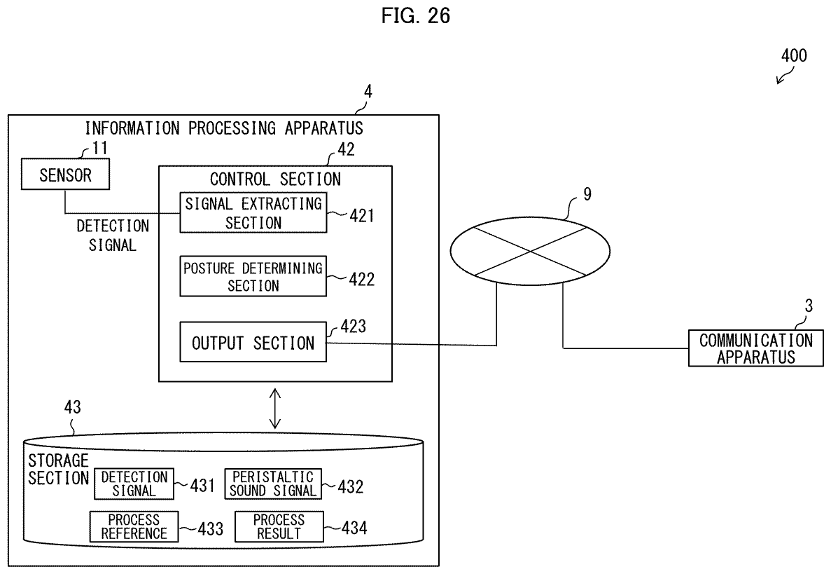

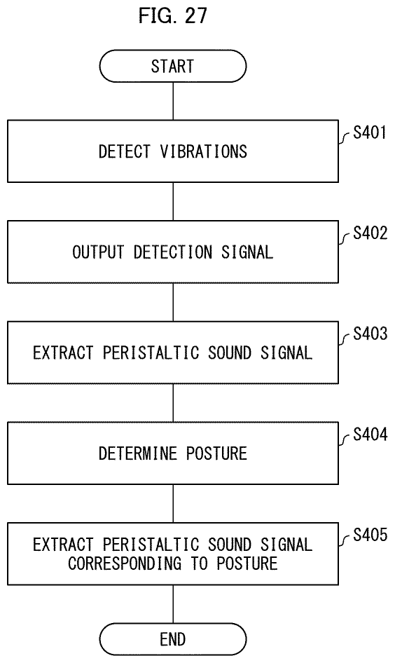

In Embodiments 1 to 4 described above, the information processing systems are each configured such that a heart sound signal and a peristaltic sound signal are extracted from a detection signal indicating vibrations which have been generated from a subject W1, the posture of the subject W1 is determined on the basis of a heartbeat-related ballistic motion signal specified from the heart sound signal, and the peristaltic sound signal corresponding to the posture of the subject W1 is extracted. However, the present invention is not limited to this configuration. For example, it is also possible to (i) determine the posture of a subject W1 from a detection signal indicating vibrations which have been generated from the subject W1 and (ii) extract a peristaltic sound signal corresponding to the posture. An information processing system 400 having such a configuration is described with reference to FIGS. 26 and 27. For convenience, members having the same functions as those of the above-described members are given the same reference signs, and descriptions thereof are not repeated.

### (Configuration of Information Processing System 400)

FIG. 26 is a functional block diagram illustrating an example configuration of the information processing system 400 in accordance with Embodiment 7. As illustrated in FIG. 26, the information processing system 400 differs from the information processing system 100 in that the information processing system 400 includes an information processing apparatus 4 instead of the information processing apparatus 1. In the other configurations, the information processing system 400 is the same as the information processing system 100.

As illustrated in FIG. 26, the information processing apparatus 4 includes a sensor 11, a control section 42, and a storage section 43.

As an example, the control section 42 may be a central processing unit (CPU). The control section 42 retrieves a control program which is software stored in the storage section 43, and loads the control program into a memory such as a random access memory (RAM) so as to control each constituent element included in the information processing apparatus 1. Note that, for simplicity of description, the control program is not illustrated in the storage section 43 illustrated in FIG. 26.

As illustrated in FIG. 26, the control section 42 includes a signal extracting section 421, a posture determining section 422, and an output section 423.

The signal extracting section 421 differs from the signal extracting section 121 in that the signal extracting section 421 extracts, from a detection signal outputted from the sensor 11, a peristaltic sound signal indicating a peristaltic sound in the stomach and intestines of a subject W1, and further extracts the peristaltic sound signal corresponding to a posture which has been determined by the posture determining section 422 (described later). The signal extracting section 421 may extract, from the detection signal outputted from the sensor 11, a heartbeat-related ballistic motion signal indicating a heartbeat-related ballistic motion of the subject W1. Note, here, that a peristaltic sound signal has a frequency characteristic of having a peak in at least one of (i) the range of 150 Hz to 350 Hz and (ii) the range of 400 Hz to 700 Hz. As an example, a heartbeat-related ballistic motion signal has a frequency characteristic of having a high signal strength in 1 Hz to 50 Hz, in particular, having a high signal strength in the frequency band of 1 Hz to 3 Hz. The signal extracting section 421 applies a known technique, such as frequency separation, to the detection signal so as to extract the peristaltic sound signal and the heartbeat-related ballistic motion signal, which respectively have the above-described frequency characteristics, from the detection signal which has been obtained.

In a case where the sensor 11 has a plurality of detection areas, the signal extracting section 421 may extract peristaltic sound signals or heartbeat-related ballistic motion signals for the respective plurality of detection areas.

The signal extracting section 421 may store, in the storage section 43, information indicating the detection signal which has been obtained from the sensor 11 and information pertaining to a process carried out by the signal extracting section 421. The information pertaining to the process carried out by the signal extracting section 421 includes information indicating the peristaltic sound signal which has been extracted from the detection signal (detection signal 431 and peristaltic sound signal 432 illustrated in FIG. 26). These signals may be stored together with time information indicating the time at which the detection signal, from which these signals have been extracted, was detected.

The posture determining section 422 determines the posture of the subject W1 on the basis of a detection signal outputted from the sensor 11. As an example, when determining the posture of the subject W1, the posture determining section 422 may determine the posture of the subject W1 by inputting, into a posture determination model, the detection signal outputted from the sensor 11. The posture determination model is a model obtained by machine learning with use of training data in which a detection signal is an explanatory variable and the posture of the subject W1 which posture corresponds to the detection signal is a response variable. A known machine learning algorithm can be applied to the machine learning for generating the posture determination model. Note that the posture determination model may be stored in the storage section 43 or may be stored in an apparatus other than the information processing apparatus 4.

In a case where the sensor 11 has a plurality of detection areas, the posture determining section 422 may determine the posture of the subject W1 on the basis of a result of comparison of signal strengths of detection signals that have been outputted from the respective plurality of detection areas.

In a case where the signal extracting section 421 extracts a heartbeat-related ballistic motion signal, the posture determining section 422 may determine the posture of the subject W1 on the basis of the heartbeat-related ballistic motion signal. As an example, when determining the posture of the subject W1, the posture determining section 422 may determine the posture of the subject W1 by inputting, into a posture determination model, the heartbeat-related ballistic motion signal which has been extracted by the signal extracting section 421. The posture determination model is a model obtained by machine learning with use of training data in which a heartbeat-related ballistic motion signal is an explanatory variable and the posture of the subject W1 which posture corresponds to the heartbeat-related ballistic motion signal is a response variable.

In a case where the sensor 11 has a plurality of detection areas, the posture determining section 422 may determine the posture of the subject W1 on the basis of a result of comparison of the frequency characteristics and/or the signal strengths of heartbeat-related ballistic motion signals which have been extracted by the signal extracting section 421 for the respective plurality of detection areas.

In a case where the sensor 11 has a plurality of detection areas, the posture determining section 422 may determine the posture of the subject W1 by inputting, into a posture determination model, heartbeat-related ballistic motion signals which have been extracted by the signal extracting section 421 for the respective plurality of detection areas. The posture determination model is a model obtained by machine learning with use of training data in which heartbeat-related ballistic motion signals, the number of which is the same as the number of the plurality of detection areas, are an explanatory variable and the posture of the subject W1 which posture corresponds to the heartbeat-related ballistic motion signals is a response variable. A known machine learning algorithm can be applied to the machine learning for generating the posture determination model. Note that the posture determination model may be stored in the storage section 43 or may be stored in an apparatus other than the information processing apparatus 4.

The posture determining section 422 may store, in the storage section 43, a result of a process carried out by the posture determining section 422. The result of the process carried out by the posture determining section 422 can be information indicating a result of determination of the posture of the subject W1 (determination result 434 illustrated in FIG. 26).

The output section 423 outputs, to an external apparatus, a detection signal outputted from the sensor 11, a result of a process carried out in the control section 42, or various pieces of information stored in the storage section 43. As an example, the output section 423 outputs, to a communication apparatus 3, (i) information indicating a detection signal outputted from the sensor 11, (ii) information indicating a peristaltic sound signal which has been extracted by the signal extracting section 421, or (iii) information indicating a result of determination of a posture which determination has been made by the posture determining section 422.

A process reference 433 which is information indicating a reference in the above-described process (e.g., extraction of signals, determination of a posture, etc.) that can be carried out by each section included in the control section 42 may be stored in the storage section 43.

### (Processes Carried Out By Information Processing System 400)

A flow of processes carried out by the information processing system 400 is described with reference to FIG. 27. FIG. 27 is a flowchart illustrating an example flow of the processes carried out by the information processing system 400 (for example, information processing apparatus 4).

As illustrated in FIG. 27, the information processing system 400 carries out processes in steps S401 to S405. S401, S402, and S405 are similar to S101, S102, and S105, respectively, and therefore descriptions thereof are omitted here.

In a case where the signal extracting section 421 of the control section 42 obtains a detection signal which has been outputted by the sensor 11 in S102, the signal extracting section 421 extracts a peristaltic sound signal from the detection signal (S403: first extracting step).

In a case where the posture determining section 422 obtains the detection signal which has been outputted by the sensor 11, the posture determining section 422 determines the posture of a subject W1 on the basis of the detection signal (S404: determining step). The posture determining section 422 outputs a result of determination of the posture to the signal extracting section 421.

Note that the timing when the information processing system 400 carries out the processes illustrated in FIG. 27 can be set as desired. For example, the information processing system 400 may carry out the processes illustrated in FIG. 27 every given period of time (for example, 1 hour), or may carry out the processes illustrated in FIG. 27 every time it is determined via the sensor 11 that the subject W1 has got out of a bed.

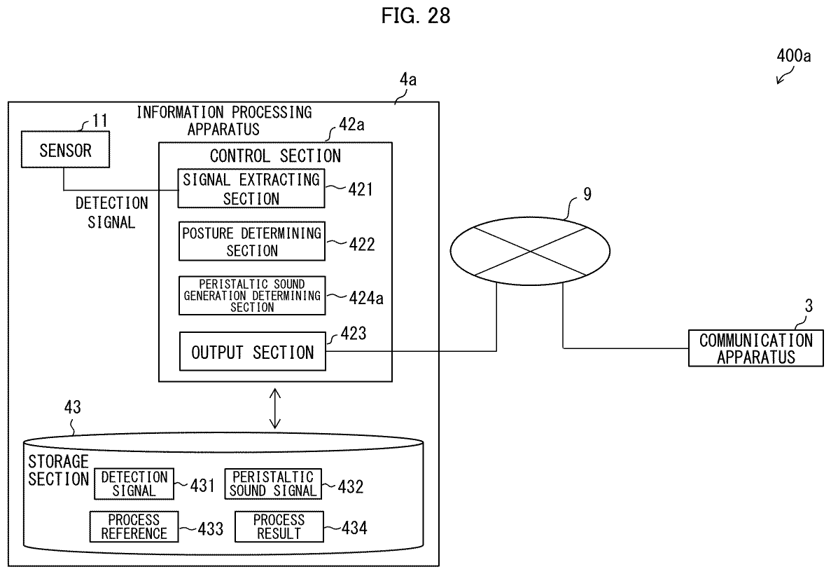

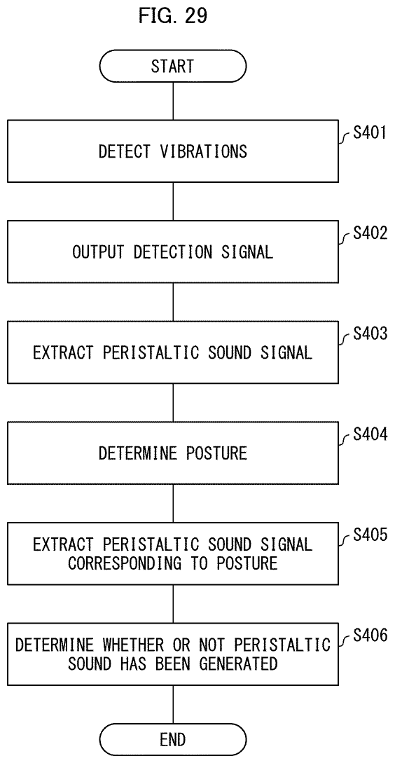

(Variation 1 of Configuration of Information Processing System 400) Variation 1 (hereinafter, referred to as “information processing system 400a”) of the configuration of the information processing system 400 is described with reference to FIGS. 28 and 29. For convenience, members having the same functions as those of the above-described members are given the same reference signs, and descriptions thereof are not repeated.
[Configuration of Information Processing System 400a]

A configuration of the information processing system 400a is described with reference to FIG. 28. FIG. 28 is a functional block diagram illustrating an example configuration of the information processing system 400a.

As illustrated in FIG. 28, the information processing system 400a differs from the information processing system 400 in that the information processing system 400a includes an information processing apparatus 4a instead of the information processing apparatus 4. In the other configurations, the information processing system 400a is the same as the information processing system 400.

As illustrated in FIG. 28, the information processing apparatus 4a differs from the information processing apparatus 4 in that the information processing apparatus 4a includes a control section 42a instead of the control section 42. The control section 42a differs from the control section 42 in that the control section 42a further includes a peristaltic sound generation determining section 424a.

The peristaltic sound generation determining section 424a determines whether or not a peristaltic sound has been generated in vibrations generated from a subject W1. As an example, the peristaltic sound generation determining section 424a determines that a peristaltic sound has been generated, in a case where a peristaltic sound signal extracted by a signal extracting section 421 has at least one of (i) a given frequency characteristic, (ii) a change in frequency characteristic at a given time, (iii) a given signal strength at a given frequency, and (iv) a change in signal strength at a given frequency at a given time.

An example of the “change in signal strength at a given frequency at a given time” can be a change such that, at a given time, a peristaltic sound signal has an increased signal strength in the frequency band of 150 to 350 Hz or 400 to 700 Hz. Note that the given time is a time which can be set as desired.

[Processes Carried Out By Information Processing System 400a]

A flow of processes carried out by the information processing system 400a is described with reference to FIG. 29. FIG. 29 is a flowchart illustrating an example flow of the processes carried out by the information processing system 400a (for example, information processing apparatus 4a).

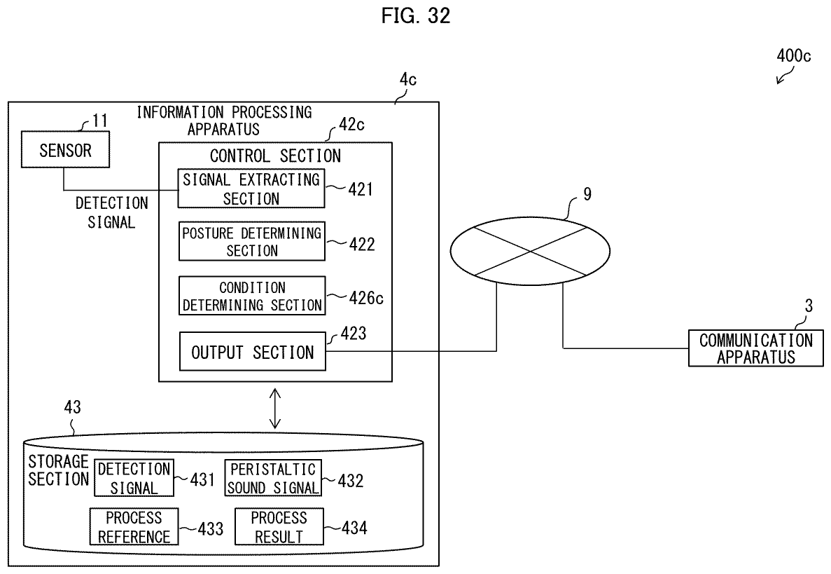

As illustrated in FIG. 29, the information processing system 400a carries out processes in steps S401 to S406. S401 to S405 are similar to the steps illustrated in FIG. 32, and therefore descriptions thereof are omitted here.

In a case where the peristaltic sound generation determining section 424a obtains a peristaltic sound signal which has been extracted by the signal extracting section 421 in S405 and which corresponds to the posture of a subject W1, the peristaltic sound generation determining section 424a determines whether or not a peristaltic sound has been generated in the stomach and intestines of the subject W1, on the basis of the peristaltic sound signal (S406). The peristaltic sound generation determining section 424a determines that a peristaltic sound has been generated, in a case where the peristaltic sound signal which has been extracted by the signal extracting section 421 and which corresponds to the posture of the subject W1 has at least one of (i) a given frequency characteristic, (ii) a change in frequency characteristic at a given time, (iii) a given signal strength at a given frequency, and (iv) a change in signal strength at a given frequency at a given time. The peristaltic sound generation determining section 424a may output a result of determination to an output section 423, and the output section 423 may output, to a communication apparatus 3, information including the result of the determination.

Note that the timing when the information processing system 400a carries out the processes illustrated in FIG. 29 can be set as desired. For example, the information processing system 400a may carry out the processes illustrated in FIG. 29 every given period of time (for example, 1 hour), or may carry out the processes illustrated in FIG. 29 every time it is determined via the sensor 11 that the subject W1 has got out of a bed.

### (Variation 2 of Configuration of Information Processing System 400)

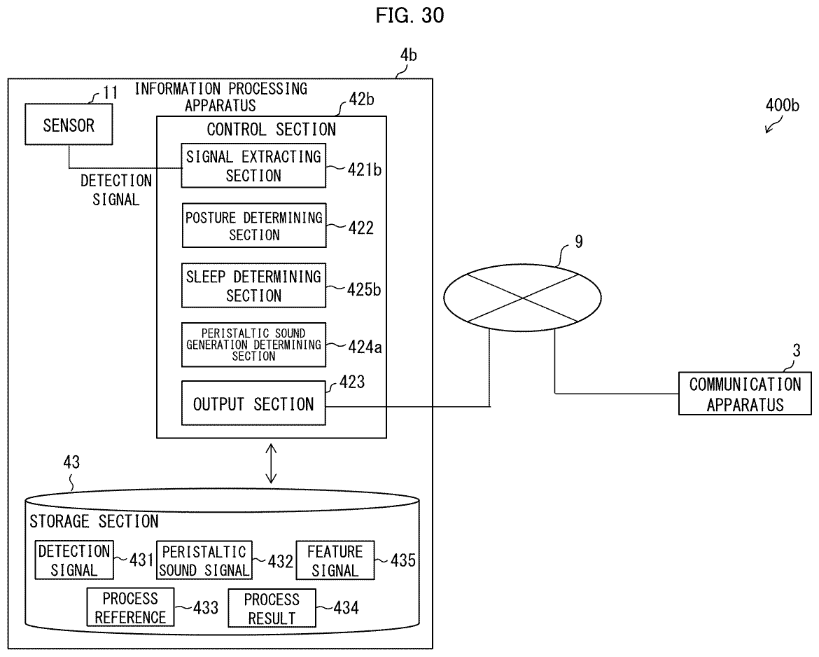

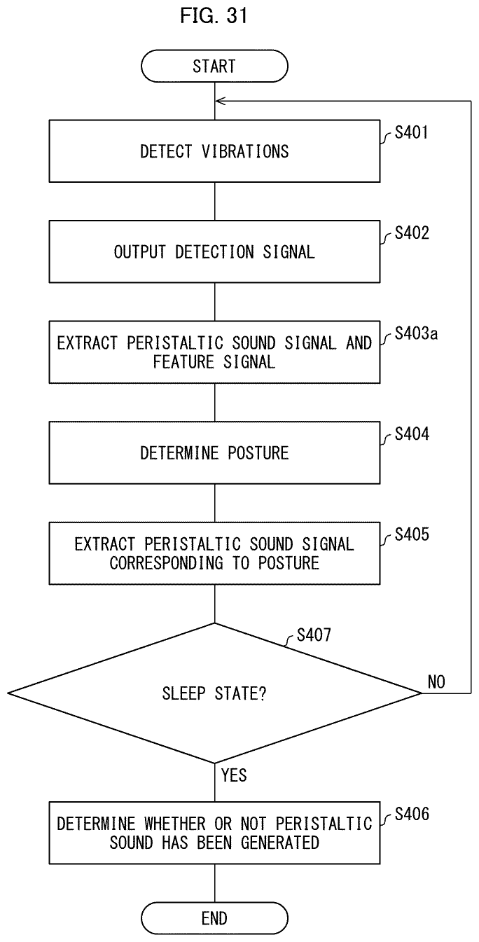

Variation 2 (hereinafter, referred to as “information processing system 400b”) of the configuration of the information processing system 400 is described with reference to FIGS. 30 and 31. For convenience, members having the same functions as those of the above-described members are given the same reference signs, and descriptions thereof are not repeated.

[Configuration of Information Processing System 400b]

A configuration of the information processing system 400b is described with reference to FIG. 30. FIG. 30 is a functional block diagram illustrating an example configuration of the information processing system 400b.

As illustrated in FIG. 30, the information processing system 400b differs from the information processing system 400a in that the information processing system 400b includes an information processing apparatus 4b instead of the information processing apparatus 4a. In the other configurations, the information processing system 400b is the same as the information processing system 400a.

As illustrated in FIG. 30, the information processing apparatus 1b differs from the information processing apparatus 4a in that the information processing apparatus 1b includes a control section 42b instead of the control section 42a. The control section 42b differs from the control section 42a in that the control section 42b includes a signal extracting section 421b instead of the signal extracting section 421 and further includes a sleep determining section 425b.

The signal extracting section 421b differs from the signal extracting section 421 in that the signal extracting section 421b further extracts a feature signal from a detection signal outputted from a sensor 11. Note, here, that the “feature signal” is similar to the feature signal illustrated in FIG. 12, and includes at least one of the signals listed as the feature signal. The signal extracting section 421b may store, in a storage section 43, the feature signal which has been extracted from the detection signal.

The sleep determining section 425b determines the sleep state of a subject W1. As an example, the sleep determining section 425b determines the sleep state of the subject W1 on the basis of the feature signal which has been extracted by the signal extracting section 421b.

When determining the sleep state of the subject W1, the sleep determining section 425b may determine the sleep state of the subject W1 as follows. That is, into a sleep determination model obtained by machine learning with use of training data in which a set constituted by, as an element(s), at least one of the above-listed signals that is included in a feature signal is an explanatory variable and the sleep state of the subject W1 which sleep state corresponds to the set is a response variable, the sleep determining section 425b inputs a set constituted by an element(s) which is/are included in the feature signal that has been extracted by the signal extracting section 421b and which is/are identical to the element(s) of the explanatory variable, thereby determining the sleep state of the subject W1. A known machine learning algorithm can be applied to the machine learning for generating the sleep determination model. Note that the sleep determination model may be stored in the storage section 43 or may be stored in an apparatus other than the information processing apparatus 4b.

[Processes Carried Out By Information Processing System 400b]

A flow of processes carried out by the information processing system 400b is described with reference to FIG. 31. FIG. 31 is a flowchart illustrating an example flow of the processes carried out by the information processing system 400b.

As illustrated in FIG. 31, the information processing system 400b carries out processes in steps S401, S402, S403b, S404, S405, S407, and S406. Descriptions of S401, S402, S404, S405, and S406 are similar to the descriptions given above, and are therefore omitted.

The signal extracting section 421b extracts a peristaltic sound signal and a feature signal from a detection signal which has been obtained from the sensor 11 (S403b). The signal extracting section 421b outputs, to the sleep determining section 425b, the detection signal and the feature signal which has been extracted.

The sleep determining section 425b determines the sleep state of a subject W1 on the basis of at least one of the detection signal and the feature signal which have been obtained from the signal extracting section 421b (S407). Specifically, the sleep determining section 425b determines whether or not the subject W1 is in the sleep state.

In the case of YES in the step S407, that is, in a case where the sleep determining section 425b determines that the subject W1 is in the sleep state, a peristaltic sound generation determining section 424a carries out the process in the step S406. In the case of NO in the step S407, that is, in a case where the sleep determining section 425b determines that the subject W1 is not in the sleep state, the flow returns to the step S401.

Note that the timing when the information processing system 400b carries out the processes illustrated in FIG. 31 can be set as desired. For example, the information processing system 400b may carry out the processes illustrated in FIG. 31 every given period of time (for example, 1 hour), or may carry out the processes illustrated in FIG. 31 every time it is determined via the sensor 11 that the subject W1 has got out of a bed.

### Embodiment 8

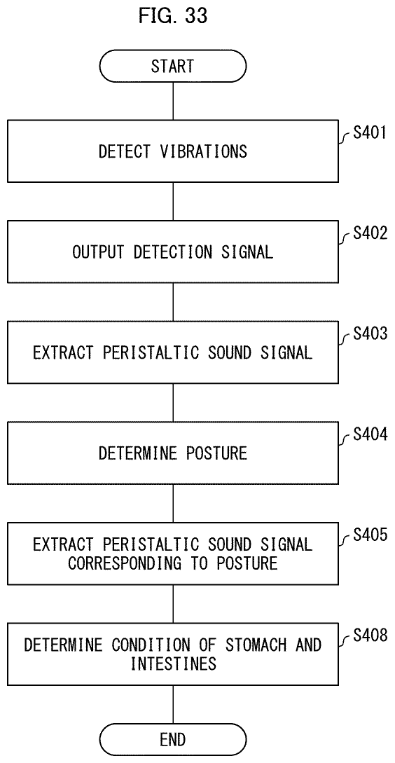

The following description will discuss Embodiment 8 of the present disclosure with reference to FIGS. 32 and 33. For convenience, members having the same functions as those of the members described in the above embodiments are given the same reference signs, and descriptions thereof are not repeated.

### (Configuration of Information Processing System 100c)

FIG. 32 is a functional block diagram illustrating an example configuration of an information processing system 400c in accordance with Embodiment 8. As illustrated in FIG. 32, the information processing system 400c differs from the information processing system 400 in that the information processing system 400c includes an information processing apparatus 4c instead of the information processing apparatus 4. In the other configurations, the information processing system 400c is the same as the information processing system 400.

As illustrated in FIG. 32, the information processing apparatus 4c differs from the information processing apparatus 4 in that the information processing apparatus 4c includes a control section 42c instead of the control section 42. The control section 42c differs from the control section 42 in that the control section 42c includes a condition determining section 426c. The condition determining section 426c is similar to the condition determining section 126c.

### (Processes Carried Out By Information Processing System 400c)

A flow of processes carried out by the information processing system 400c is described with reference to FIG. 33. FIG. 33 is a flowchart illustrating an example flow of the processes carried out by the information processing system 400c.

As illustrated in FIG. 33, the information processing system 400c carries out processes in steps S401 to S405 and S408. Descriptions of the steps S401 to S405 are similar to the descriptions given above, and a description of the step S408 is similar to the description of the step S108.

### Embodiment 9

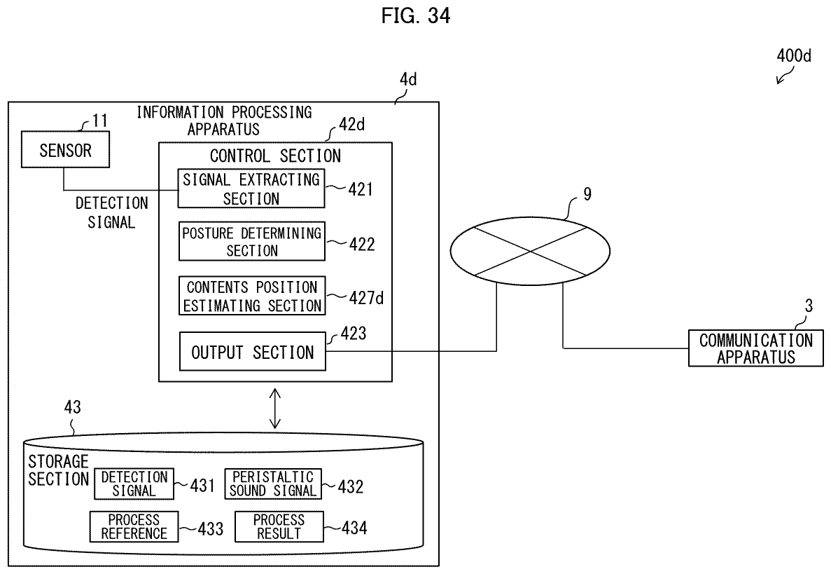

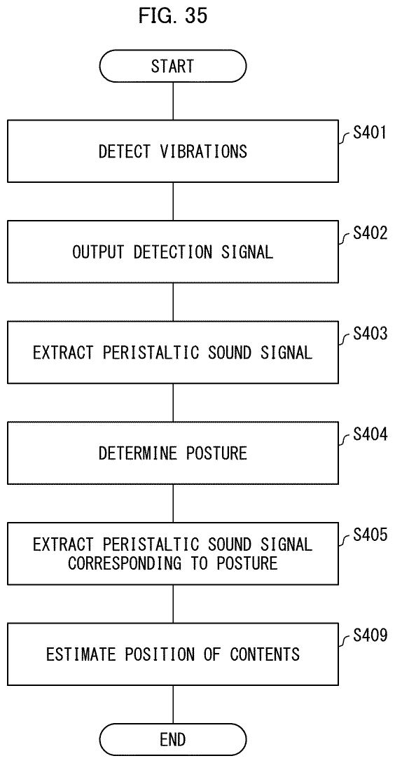

The following description will discuss Embodiment 9 of the present disclosure with reference to FIGS. 34 and 35. For convenience, members having the same functions as those of the members described in the above embodiments are given the same reference signs, and descriptions thereof are not repeated.

### (Configuration of Information Processing System 400d)

An information processing system 400d which is configured such that a sensor 11 includes a plurality of detection areas and outputs detection signals detected in the respective plurality of detection areas is described with reference to FIG. 34.

FIG. 34 is a functional block diagram illustrating an example configuration of the information processing system 400d in accordance with Embodiment 9. As illustrated in FIG. 34, the information processing system 400d differs from the information processing system 400 in that the information processing system 400d includes an information processing apparatus 4d instead of the information processing apparatus 4. In the other configurations, the information processing system 400d is the same as the information processing system 400.

As illustrated in FIG. 34, the information processing apparatus 4d differs from the information processing apparatus 1 in that the information processing apparatus 4d includes a control section 42d instead of the control section 42. The control section 42d differs from the control section 42 in that the control section 42d further includes a contents position estimating section 427d. The contents position estimating section 427d is similar to the contents position estimating section 127d.

### (Processes Carried Out By Information Processing System 400d)

A flow of processes carried out by the information processing system 400d is described with reference to FIG. 35. FIG. 35 is a flowchart illustrating an example flow of the processes carried out by the information processing system 400d.

As illustrated in FIG. 35, the information processing system 400d carries out processes in steps S401 to S405 and S409. Descriptions of the steps S401 to S405 are similar to the descriptions given above, and a description of the step S409 is similar to the description of the step S109.

### Embodiment 10

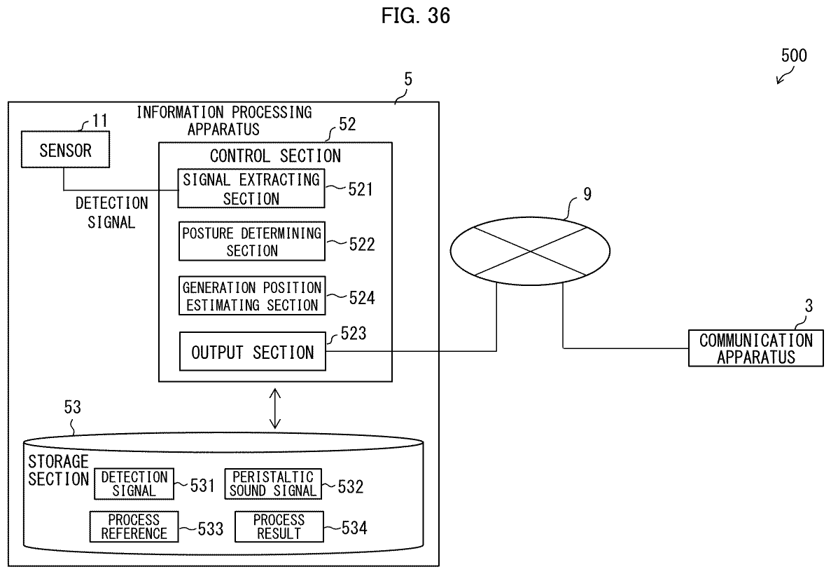

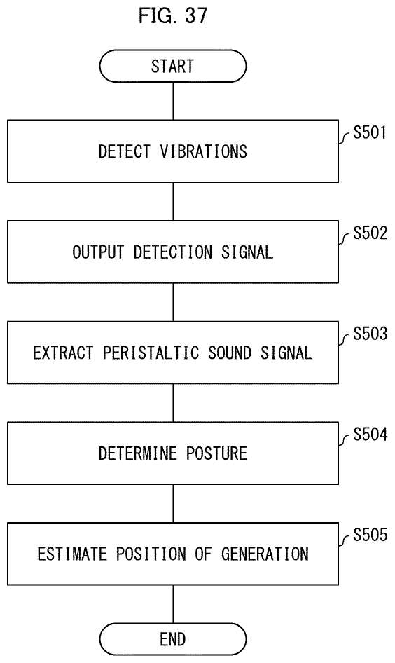

As in the following embodiment, an information processing system in accordance with the present invention may be configured such that the posture of a subject W1 is determined from a detection signal indicating vibrations which have been generated from the subject W1, a peristaltic sound signal corresponding to the posture is extracted, and then the position of generation of a peristaltic sound indicated by the peristaltic sound signal is further estimated. An information processing system 500 having such a configuration is described with reference to FIGS. 36 and 37. For convenience, members having the same functions as those of the above-described members are given the same reference signs, and descriptions thereof are not repeated.

### (Outline of Information Processing System 500)

In Embodiments 4 to 6 described above, the information processing systems are each configured such that a heart sound signal and a peristaltic sound signal are extracted from a detection signal indicating vibrations which have been generated from a subject W1, the posture of the subject W1 is determined on the basis of a heartbeat-related ballistic motion signal specified from the heart sound signal, the peristaltic sound signal corresponding to the posture of the subject W1 is extracted, and the position of generation of a peristaltic sound indicated by the peristaltic sound signal is estimated on the basis of the peristaltic sound signal which has been extracted and the posture which has been determined. However, the present invention is not limited to this configuration. For example, it is also possible to determine the posture of a subject W1 from a detection signal indicating vibrations which have been generated from the subject W1, extract a peristaltic sound signal corresponding to the position, and estimate the position of generation of a peristaltic sound indicated by the peristaltic sound signal on the basis of the peristaltic sound signal which has been extracted and the posture which has been determined. An information processing system 400 having such a configuration is described with reference to FIGS. 31 and 32. For convenience, members having the same functions as those of the above-described members are given the same: reference signs, and descriptions thereof are not repeated.

### (Configuration of Information Processing System 500)

FIG. 36 is a functional block diagram illustrating an example configuration of the information processing system 500 in accordance with Embodiment 10. As illustrated in FIG. 36, the information processing system 500 differs from the information processing system 200 in that the information processing system 500 includes an information processing apparatus 5 instead of the information processing apparatus 2. In the other configurations, the information processing system 500 is the same as the information processing system 200.

As illustrated in FIG. 36, the information processing apparatus 5 differs from the information processing apparatus 2 in that the information processing apparatus 5 includes a control section 52 and a storage section 53 instead of the control section 22 and the storage section 23, respectively. The control section 52 includes a signal extracting section 521, a posture determining section 522, a generation position estimating section 524, and an output section 523. The signal extracting section 521, the posture determining section 522, and the output section 523 are similar to the signal extracting section 421, the posture determining section 422, and the output section 423, respectively, and the storage section 53 is similar to the storage section 43. Thus, descriptions thereof are omitted here.

The generation position estimating section. 524 estimates the position of generation of a peristaltic sound indicated by a peristaltic sound signal which has been extracted by the signal extracting section 421, on the basis of the peristaltic sound signal and a posture which has been determined by the posture determining section 422.

### (Processes Carried Out By Information Processing System 500)

A flow of processes carried out by the information processing system 500 is described with reference to FIG. 37. FIG. 37 is a flowchart illustrating an example flow of the processes carried out by the information processing system 500.

As illustrated in FIG. 37, the information processing system 500 carries out processes in steps S501 to S505. S501, S502, and S505 are similar to S201, S202, and S205, respectively, and therefore descriptions thereof are omitted here.

In a case where the signal extracting section 521 of the control section 52 obtains a detection signal which has been outputted by a sensor 11 in S502, the signal extracting section 521 extracts a peristaltic sound signal from the detection signal (S503: first extracting step).

In a case where the posture determining section 522 obtains the detection signal which has been outputted by the sensor 11, the posture determining section 522 determines the posture of a subject W1 on the basis of the detection signal (S504: determining step). The posture determining section 522 outputs a result of determination of the posture to the signal extracting section 521.

The generation position estimating section 524 estimates the position of generation of a peristaltic sound indicated by the peristaltic sound, on the basis of (i) the result of the determination which result has been obtained from the posture determining section 522 and (ii) the peristaltic sound signal which has been obtained from the signal extracting section 521 (S505: estimating step). The generation position estimating section 524 may output a result of estimation to the output section 523, and the output section 523 may output, to a communication apparatus 3, information including the result of the estimation.

### (Variation of Configuration of Information Processing System 500)

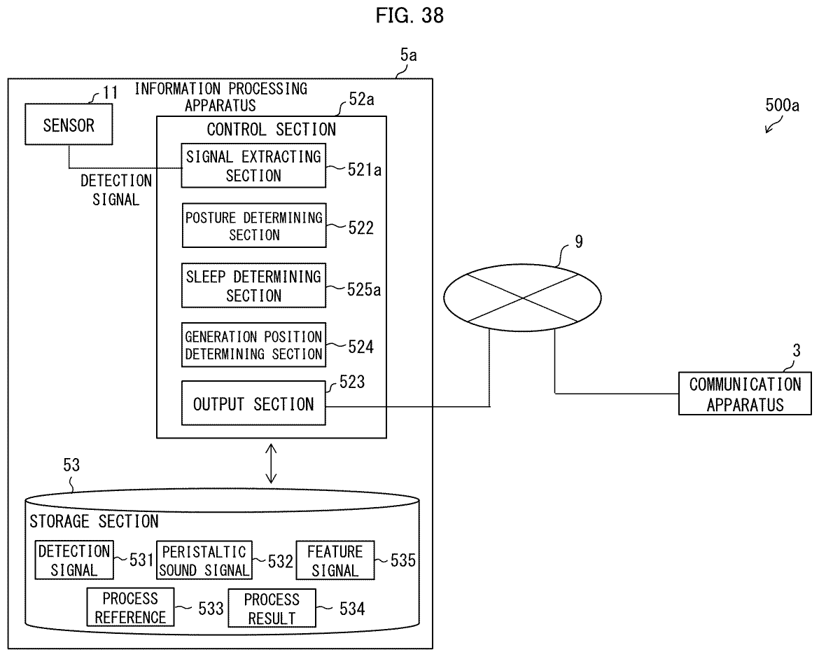

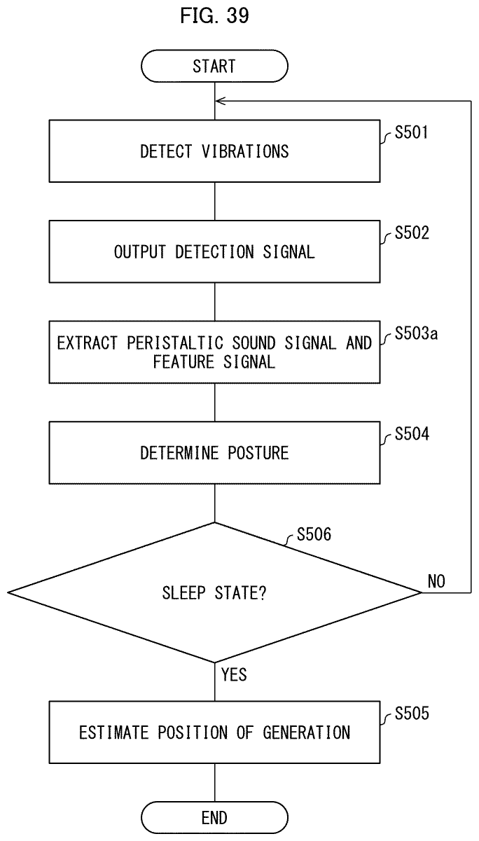

A variation (hereinafter, referred to as “information processing system 500a”) of the configuration of the information processing system 500 is described with reference to FIGS. 38 and 39. For convenience, members having the same functions as those of the above-described members are given the same reference signs, and descriptions thereof are not repeated.

[Configuration of Information Processing System 500a]

A configuration of the information processing system 500a is described with reference to FIG. 38. FIG. 38 is a functional block diagram illustrating an example configuration of the information processing system 500a.

As illustrated in FIG. 38, the information processing system 500a differs from the information processing system 500 in that the information processing system 500a includes an information processing apparatus 5a instead of the information processing apparatus 5. In the other configurations, the information processing system 500a is the same as the information processing system 500.

As illustrated in FIG. 38, the information processing apparatus 5a differs from the information processing apparatus 5 in that the information processing apparatus 5a includes a control section 52a instead of the control section 52. The control section 52a differs from the control section 52 in that the control section 52a includes a signal extracting section 521a instead of the signal extracting section 521 and further includes a sleep determining section 525a.

The signal extracting section 521a is similar to the above-described signal extracting section 421b, and the sleep determining section 525a is similar to the above-described sleep determining section 425b. Thus, descriptions thereof are omitted here.

[Processes Carried Out By Information Processing System 500a]

A flow of processes carried out by the information processing system 500a is described with reference to FIG. 39. FIG. 39 is a flowchart illustrating an example flow of the processes carried out by the information processing system 500a.

As illustrated in FIG. 39, the information processing system 500a carries out processes in steps S501, S502, S503a, S504, S506, and S505. Descriptions of S501, S502, S504, and S505 are similar to the descriptions given above, and descriptions of S503a and S506 are respectively similar to the descriptions of S203a and S206.

### Embodiment 11

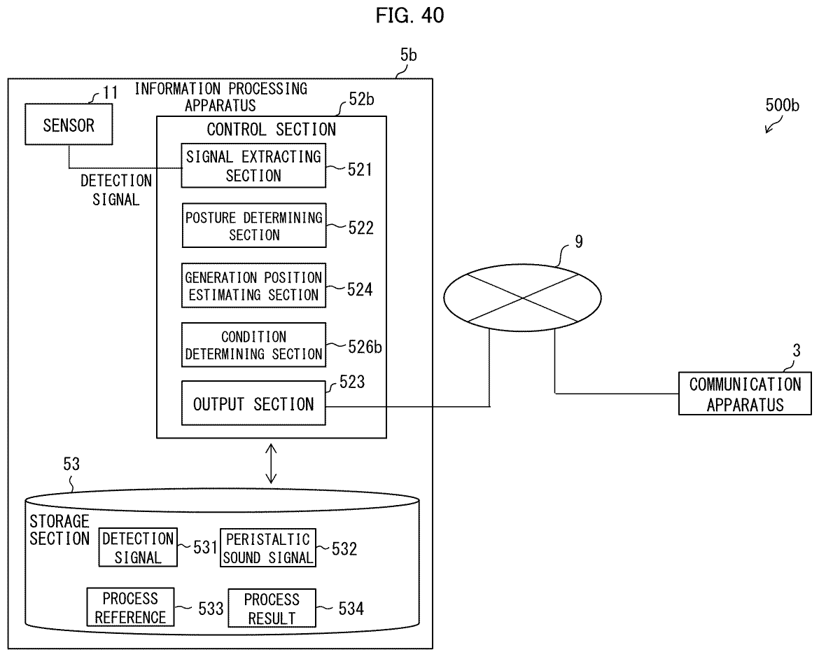

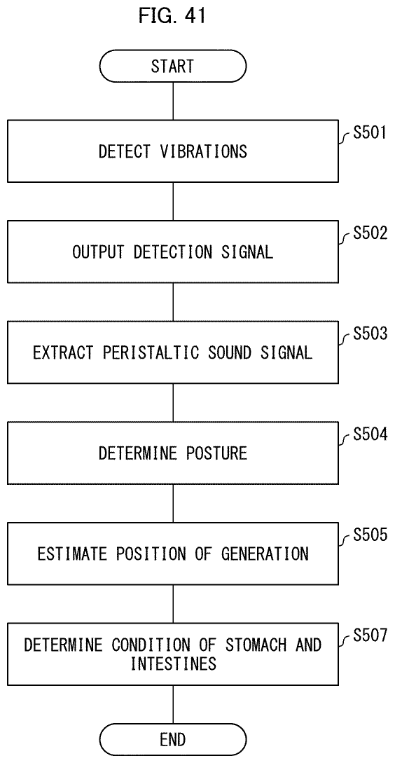

The following description will discuss Embodiment 11 of the present disclosure with reference to FIGS. 40 and 41. For convenience, members having the same functions as those of the members described in the above embodiments are given the same reference signs, and descriptions thereof are not repeated.

### (Configuration of Information Processing System 500b)

FIG. 40 is a functional block diagram illustrating an example configuration of the information processing system 500b in accordance with Embodiment 11. As illustrated in FIG. 40, the information processing system 500b differs from the information processing system 500 in that the information processing system 500b includes an information processing apparatus 5b instead of the information processing apparatus 5. In the other configurations, the information processing system 500b is the same as the information processing system 500.

As illustrated in FIG. 40, the information processing apparatus 5b differs from the information processing apparatus 5 in that the information processing apparatus 5b includes a control section 52b instead of the control section 52. The control section 52b differs from the control section 52 in that the control section 52b further includes a condition determining section 526b. The condition determining section 526b is similar to the condition determining section 126c.

### (Processes Carried Out By Information Processing System 500b)

A flow of processes carried out by the information processing system 500b is described with reference to FIG. 41. FIG. 41 is a flowchart illustrating an example flow of the processes carried out by the information processing system 500b.

As illustrated in FIG. 41, the information processing system 500b carries out processes in steps S501 to S505 and S507. Descriptions of the steps S501 to S505 are similar to the descriptions given above, and a description of S507 is similar to the description of S207.

### Embodiment 12

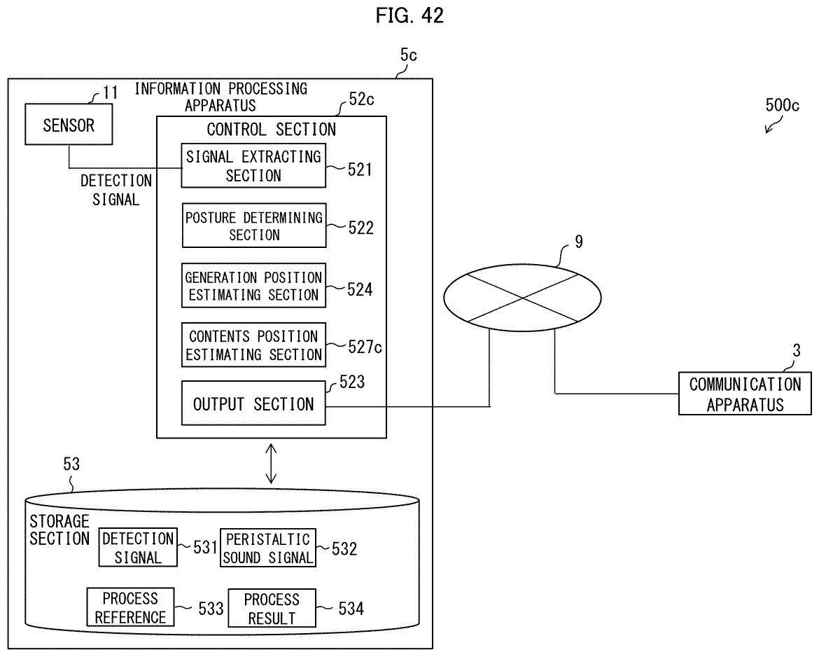

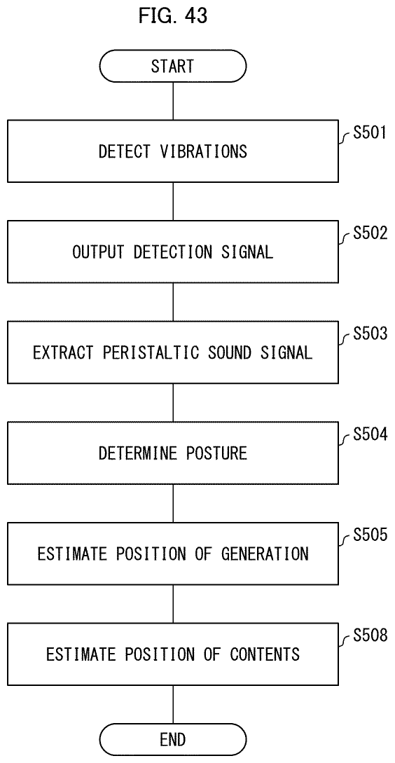

The following description will discuss Embodiment 12 of the present disclosure with reference to FIGS. 42 and 43. For convenience, members having the same functions as those of the members described in the above embodiments are given the same reference signs, and descriptions thereof are not repeated.

### (Configuration of Information Processing System 500b)

FIG. 42 is a functional block diagram illustrating an example configuration of an information processing system 500c in accordance with Embodiment 12. As illustrated in FIG. 42, the information processing system 500c differs from the information processing system 500 in that the information processing system 500c includes an information processing apparatus 5c instead of the information processing apparatus 5. In the other configurations, the information processing system 500c is the same as the information processing system 500.

As illustrated in FIG. 42, the information processing apparatus 5c differs from the information processing apparatus 5 in that the information processing apparatus 5c includes a control section 52c instead of the control section 52. The control section 52c differs from the control section 52 in that the control section 52c further includes a contents position estimating section 527c. The contents position estimating section 527c is similar to the contents position estimating section 127d.

### (Processes Carried Out By Information Processing System 500c)

A flow of processes carried out by the information processing system 500c is described with reference to FIG. 43. FIG. 43 is a flowchart illustrating an example flow of the processes carried out by the information processing system 500c.

As illustrated in FIG. 43, the information processing system 500c carries out processes in steps S501 to S505 and S508. Descriptions of the steps S501 to S505 are similar to the descriptions given above, and a description of S508 is similar to the description of S208.

### (Example Display)

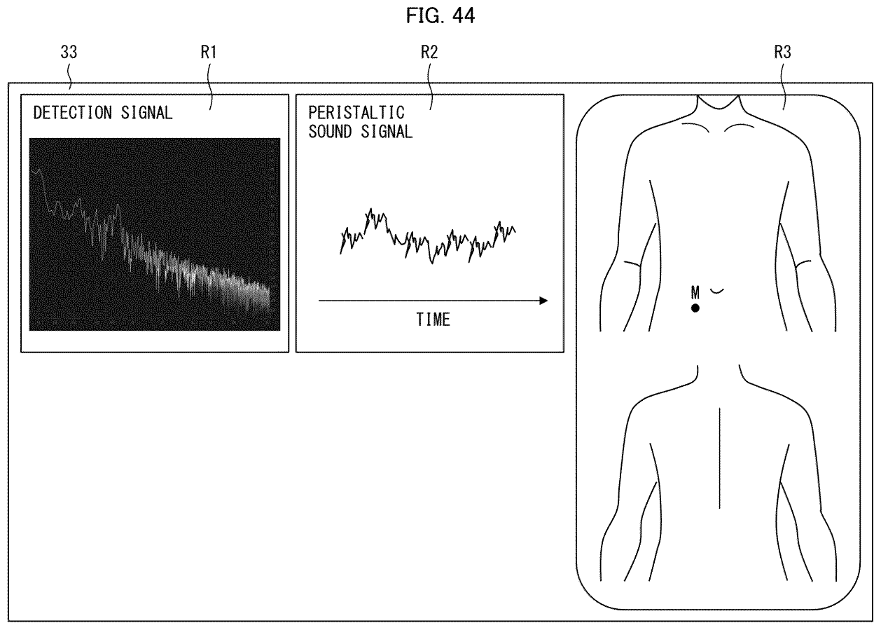

A specific example of a display screen displayed by the display section 34 of the communication apparatus 3 in each embodiment above is described with reference to FIG. 44. FIG. 44 illustrates an example display screen displayed by the display section 34 of each of the information processing systems 100,100a to 100d, 200, 200a to 200c, 400, 400a to 400d, 500, and 500a to 500c.

As illustrated in FIG. 44, the display section 34 may display a region R1 in which waveform data of a detection signal outputted by the sensor 11 which has detected vibrations of the subject W1 is displayed, a region R2 in which waveform data of a peristaltic sound signal which has been extracted by the signal extracting section 121 is displayed, and a region R3 in which a result of estimation of the position M of generation of a peristaltic sound is displayed.

Although an example display screen displayed in each of the information processing systems 100,100a to 100d, 200, 200a to 200c, 400, 400a to 400d, 500, and 500a to 500c is illustrated, the display screen is not limited to this example. All pieces of information pertaining to the processes carried out in each embodiment may be displayed, or arbitrarily selected information may be displayed.

### [Variations]

A member which carries out each of the processes described in the above embodiments may be any member, and is not limited to the above example. For example, the processes carried out by the control section may be carried out by one or more information processing apparatuses. That is, the processes carried out by the control section may be all carried out by a single information processing apparatus or may be shared by a plurality of information processing apparatuses.

The configurations included in the control sections described in the above embodiments can be recombined as desired, and are not limited to the above examples. For example, an information processing apparatus constituting an information processing system may be configured to include a condition determining section that determines the condition of the stomach and intestines of a subject W1 and a contents position estimating section that estimates the position of contents.

### [Machine Learning For Generating Posture Determination Model, Sleep Determination Model, Condition Determination Model, And Position Estimation Model]

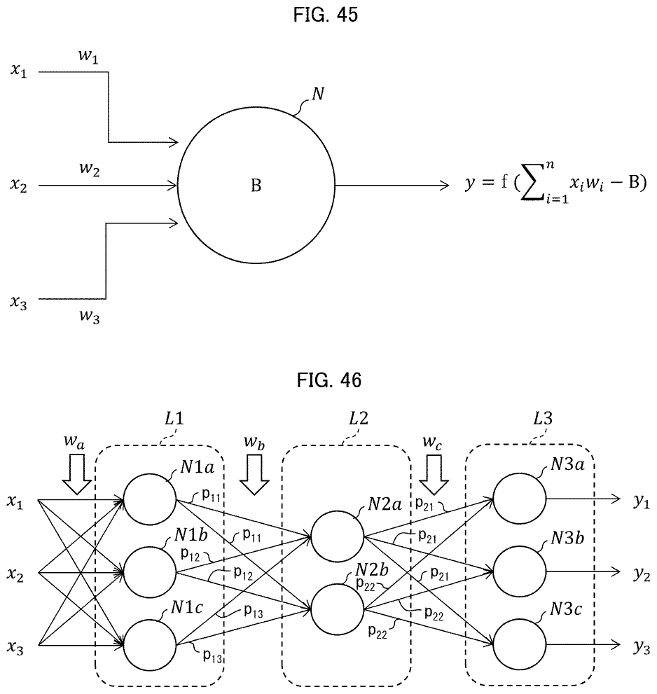

A known machine learning algorithm such as a neural network or a support vector machine can be applied to the machine learning for generating the posture determination model, the sleep determination model, the condition determination model, and the position estimation model. Here, a case where a neural network is used for the posture determination model, the sleep determination model, the condition determination model, and the position estimation model is described with reference to FIGS. 44 and 46. The neural network is constituted by, for example, a processor, a memory, and the like that realize a neural network mimicking a model of a neuron illustrated in FIG. 45. FIG. 45 is a schematic view for explaining the model of the neuron, and FIG. 46 is for explaining the neural network.

The neural network is constituted by, for example, an input layer that includes a plurality of neurons, a hidden layer (intermediate layer) that includes a plurality of neurons, and an output layer that includes a plurality of neurons. A neuron outputs a result y with respect to a plurality of inputs x, as illustrated in FIG. 45. Each of the inputs x is multiplied by a corresponding weighting factor w. For example, in FIG. 45, an input x₁ is multiplied by a weighting factor w₁, an input x₂ is multiplied by a weighting factor w₂, and an input x₃ is multiplied by a weighting factor w₃. The neuron sums up results obtained by multiplying each of the inputs by the corresponding weighting factor, and outputs the result y by substituting, into an activation function f, a result obtained by considering a bias B with respect to a summed result.

Next, the neural network obtained by combining the neurons is described with reference to FIG. 46. FIG. 46 is a schematic view illustrating the neural network that has an input layer L1, a hidden layer L2, and an output layer L3. In the neural network illustrated in FIG. 46, a plurality of inputs x are inputted into the input layer L1, and results y are outputted from the output layer L3. The neural network may has a plurality of hidden layers. In FIG. 46, for example, each of inputs x₁ to x₃ is multiplied by a corresponding weighting factor wₐ. Results obtained by such multiplication are each inputted into each of three neurons N1a to N1c. Note, here, that the neurons N1a to N1c respectively output p₁₁ to p₁₃. Vectors (p₁₁, p₁₂, and p₁₃) can be respectively regarded as feature vectors obtained by extracting features of input vectors (x₁, x₂, and x₃). These feature vectors (p₁₁, p₁₂, and p₁₃) are feature vectors between the input layer L1 and the hidden layer L2.

Each of p₁₁ to p₁₃ is multiplied by a corresponding weighting factor w_{b}. Results obtained by such multiplication are each inputted into each of two neurons N2a and N2b. Note, here, that the neurons N2a and N2b respectively output p₂₁ and p₂₂. These vectors (p₂₁ and p₂₂) are feature vectors between the hidden layer L2 and the output layer L3.

Each of p₂₁ and p₂₂ is multiplied by a corresponding weighting factor w_{c}. Results obtained by such multiplication are each inputted into each of three neurons N3a to N3c. The neurons N3a to N3c respectively output results y₁ to y₃.

Operations of the neural network include an operation in a training mode and an operation in an estimation mode. In the training mode, the neural network trains (adjusts) a parameter that indicates the weighting factor w with use of training data including an explanatory variable and a response variable. In the estimation mode, the neural network outputs a result estimated from inputted data (e.g., feature signal) with use of the parameter adjusted by training.

In the training mode, an error between (i) a result outputted from the output layer L3 in a case where the explanatory variable included in the training data is inputted into the input layer L1 and (ii) the response variable corresponding to the explanatory variable is calculated, and the parameter is adjusted so that this error is reduced.

To adjustment of the parameter, any known method can be applied. For example, a backpropagation method may be applied. Until the error falls within a given range or until all explanatory variables included in the training data are inputted, the adjustment of the parameter may be repeated.

### [Software Implementation Example]

The functions of the information processing systems 100, 100a to 100d, 200, 200a to 200c, 400, 400a to 400d, 500, and 500a to 500c (hereinafter, referred to as “systems”) and the information processing apparatuses 1, 1a to 1d, 2, 2a to 2c, 4, 4a to 4d, 5, and 5a to 5c (hereinafter, referred to as “apparatuses”) can be implemented by a program for causing a computer to function as the systems and the apparatuses, the program causing the computer to function as each of the control blocks (in particular, each of the sections included in the control sections) of the systems and the apparatuses.

In this case, the apparatuses include a computer which has at least one control device (e.g. processor) and at least one storage device (e.g., memory), which are hardware for executing the program. The program is executed via this control device and this storage device, so that each of the functions described in the above embodiments is implemented.

The program may be recorded on one or more non-transitory computer-readable recording media. The recording media may or may not be included in the above apparatuses. In the latter case, the program may be made available to the apparatuses via any wired or wireless transmission medium.

Further, some or all of the functions of the control blocks can be implemented by a logic circuit. For example, an integrated circuit in which a logic circuit that functions as each of the control blocks is formed is also within the scope of the present invention. As another example, the functions of the control blocks can be implemented by a quantum computer.

Further, each of the processes described in the above embodiments may be carried out by artificial intelligence (AI). In this case, the AI may operate in the control device, or may operate in another device (e.g. an edge computer or a cloud server).

### [Additional Remark 1]

The present invention is not limited to the embodiments, but can be altered by a skilled person in the art within the scope of the claims. The present invention also encompasses, in its technical scope, any embodiment derived by combining technical means disclosed in differing embodiments.

Aspects of the present invention can also be expressed as follows:

In order to attain the above object, an information processing system in accordance with Aspect 1 of the present disclosure includes: a sensor that is disposed at a given position at which the sensor does not touch a subject and that detects vibrations which have been generated from the subject; a signal extracting section that extracts, from a detection signal outputted from the sensor, a peristaltic sound signal indicating a peristaltic sound in the stomach and intestines of the subject; and a posture determining section that determines a posture of the subject on the basis of the detection signal, wherein the signal extracting section extracts the peristaltic sound signal having a signal strength corresponding to the posture which has been determined.

A peristaltic sound is a sound that is generated during a peristaltic movement in which the digestive tract contracts so as to propel contents (e.g., food, gas, etc. which have been ingested). In a case where some abnormality is occurring in the stomach and intestines of a subject, a peristaltic sound in the subject may differ from a peristaltic sound in a normal condition. As an example, a peristaltic sound which differs from a peristaltic sound in a normal condition has an increase or decrease in frequency of generation, a change in sound quality, a change in strength, and the like. A generated peristaltic sound varies depending on the type of an abnormality occurring in the stomach and intestines. Therefore, it is possible to accurately estimate the condition of the stomach and intestines of a subject on the basis of a peristaltic sound in the subject.

With the above configuration, the information processing system extracts a peristaltic sound signal from a detection signal outputted from the sensor that detects vibrations which have been generated from a subject. The information processing system determines the posture of the subject on the basis of the detection signal, and extracts the peristaltic sound signal having a signal strength corresponding to the posture which has been determined.

This makes it possible for the information processing system to extract, in a peristaltic sound signal indicating a peristaltic sound included in vibrations which have been generated from a subject, only the peristaltic sound signal having a signal strength corresponding to the posture of the subject. That is, it is possible to appropriately measure a peristaltic sound regardless of the posture of a subject during measurement.

An information processing system in accordance with Aspect 2 of the present disclosure includes: a sensor that is disposed at a given position at which the sensor does not touch a subject and that detects vibrations which have been generated from the subject; a signal extracting section that extracts, from a detection signal outputted from the sensor, a heart sound signal indicating a heart sound of the subject and a peristaltic sound signal indicating a peristaltic sound in the stomach and intestines of the subject; and a posture determining section that determines a posture of the subject on the basis of a heartbeat-related ballistic motion signal that is specified from the heart sound signal which has been extracted and that indicates a heartbeat-related ballistic motion, wherein the signal extracting section extracts the peristaltic sound signal having a signal strength corresponding to the posture which has been determined.

With the above configuration, the information processing system extracts a heart sound signal and a peristaltic sound signal from a detection signal outputted from the sensor that detects vibrations which have been generated from a subject. The information processing system specifies a heartbeat-related ballistic motion signal from the heart sound signal, determines the posture of the subject on the basis of the heartbeat-related ballistic motion signal which has been specified, and extracts the peristaltic sound signal having a signal strength corresponding to the posture which has been determined. This brings about an effect similar to that brought about by the information processing system in accordance with Aspect 1.

An information processing system in accordance with Aspect 3 of the present disclosure includes: a sensor that is disposed at a given position at which the sensor does not touch a subject and that detects vibrations which have been generated from the subject; a signal extracting section that extracts, from a detection signal outputted from the sensor, a peristaltic sound signal indicating a peristaltic sound in the stomach and intestines of the subject; a posture determining section that determines a posture of the subject on the basis of the detection signal; and a generation position estimating section that estimates a position of generation of the peristaltic sound indicated by the peristaltic sound signal, on the basis of the peristaltic sound signal which has been extracted and the posture which has been determined.

With the above configuration, the information processing system extracts a peristaltic sound signal from a detection signal outputted from the sensor that detects vibrations which have been generated from a subject. The information processing system determines a posture on the basis of the detection signal, and estimates the position of generation of a peristaltic sound on the basis of the peristaltic sound signal which has been extracted and the posture which has been determined.

This makes it possible for the information processing system estimate the position of generation of a peristaltic sound included in vibrations which have been generated from a subject. For example, in a case where a peristaltic sound signal which has been extracted indicates an abnormality in the stomach and intestines, it is possible to for a medical expert to provide an appropriate treatment, considering that the abnormality is occurring at the position of generation of a peristaltic sound.

An information processing system in accordance with Aspect 4 of the present disclosure includes: a sensor that is disposed at a given position at which the sensor does not touch a subject and that detects vibrations which have been generated from the subject; a signal extracting section that extracts, from a detection signal outputted from the sensor, a heart sound signal indicating a heart sound of the subject and a peristaltic sound signal indicating a peristaltic sound in the stomach and intestines of the subject; a posture determining section that determines a posture of the subject on the basis of a heartbeat-related ballistic motion signal that is specified from the heart sound signal which has been extracted and that indicates a heartbeat-related ballistic motion; and a generation position estimating section that estimates a position of generation of the peristaltic sound indicated by the peristaltic sound signal, on the basis of the peristaltic sound signal which has been extracted and the posture which has been determined.

With the above configuration, the information processing system extracts a heart sound signal and a peristaltic sound signal from a detection signal outputted from the sensor that detects vibrations which have been generated from a subject. The information processing system determines a posture on the basis of a heartbeat-related ballistic motion signal specified from the heart sound signal which has been extracted, and estimates the position of generation of a peristaltic sound on the basis of the peristaltic sound signal which has been extracted and the posture which has been determined. This brings about an effect similar to that brought about by the information processing system in accordance with Aspect 3.

The information processing system in accordance with Aspect 5 of the present disclosure may be configured to further include a peristaltic sound generation determining section that determines whether or not the peristaltic sound has been generated, wherein the peristaltic sound generation determining section determines that the peristaltic sound has been generated, in a case where the peristaltic sound signal which has been extracted has at least one of (i) a given frequency characteristic, (ii) a change in frequency characteristic at a given time, (iii) a given signal strength at a given frequency, and (iv) a change in signal strength at a given frequency at a given time.

With the above configuration, the information processing system determines that a peristaltic sound has been generated, in a case where a peristaltic sound signal of a subject has at least one of (i) a frequency characteristic, (ii) a signal strength, (iii) a change in frequency characteristic at a given time, (iv) a given signal strength at a given frequency, and (v) a change in signal strength at a given frequency at a given time. This makes it possible for the information processing system to accurately determine a peristaltic sound included in vibrations which have been generated from a subject.

The information processing system in accordance with Aspect 6 of the present disclosure may be configured such that the signal extracting section further extracts, from the detection signal, a heartbeat-related ballistic motion signal indicating a heartbeat-related ballistic motion of the subject, and the posture determining section determines the posture of the subject on the basis of the heartbeat-related ballistic motion signal.

With the above configuration, the information processing system extracts a heartbeat-related ballistic motion signal from a detection signal outputted from the sensor that detects vibrations which have been generated from a subject. The information processing system determines the posture of the subject on the basis of the heartbeat-related ballistic motion signal which has been extracted, and extracts a peristaltic sound signal having a signal strength corresponding to the posture which has been determined. This brings about an effect similar to that brought about by the information processing system in accordance with Aspect 1.

The information processing system in accordance with Aspect 7 of the present disclosure may be configured such that the sensor has a plurality of detection areas that output respective detection signals, and the posture determining section determines the posture of the subject on the basis of a result of comparison of signal strengths of the detection signals that have been outputted from the respective plurality of detection areas.

With the above configuration, the information processing system extracts peristaltic sound signals from detection signals outputted from the respective plurality of detection areas, and determines the posture of a subject on the basis of the signal strengths of the peristaltic sound signals which have been extracted for the respective plurality of areas. This makes it possible for the information processing system to accurately determine the posture of a subject at the time when the sensor has detected vibrations generated from the subject.

The information processing system in accordance with Aspect 8 of the present disclosure may be configured such that the signal extracting section extracts, from the detection signal as the peristaltic sound signal, at least one of (i) a signal having a peak in 150 Hz to 350 Hz and (ii) a signal having a peak in 400 Hz to 700 Hz.

With the above configuration, it is possible for the information processing system to extract a peristaltic sound signal from a detection signal on the basis of a frequency characteristic.

The information processing system in accordance with Aspect 9 of the present disclosure may be configured such that the posture determining section determines the posture of the subject by inputting, into a posture determination model, the detection signal which has been specified, the posture determination model being a model obtained by machine learning with use of training data in which the detection signal is an explanatory variable and the posture of the subject is a response variable.

With the above configuration, it is possible for the information processing system to determine the posture of a subject more accurately by using the model obtained by machine learning.

The information processing system in accordance with Aspect 10 of the present disclosure may be configured such that the sensor has a plurality of detection areas that output respective detection signals, the signal extracting section extracts, for the respective plurality of detection areas, heartbeat-related ballistic motion signals each indicating a heartbeat-related ballistic motion of the subject, and the posture determining section determines the posture of the subject on the basis of a result of comparison of frequency characteristics and/or signal strengths of the heartbeat-related ballistic motion signals which have been extracted for the respective detection areas.

With the above configuration, the information processing system extracts heartbeat-related ballistic motion signals from detection signals outputted from the respective plurality of detection areas, and determines the posture of a subject on the basis of the frequency characteristics and/or the signal strengths of the heartbeat-related ballistic motion signals which have been extracted for the respective plurality of detection areas. This makes it possible for the information processing system to more accurately determine the posture of a subject at the time when the sensor has detected vibrations generated from the subject.

The information processing system in accordance with Aspect 11 of the present disclosure may be configured such that the sensor has a plurality of detection areas that output respective detection signals, the signal extracting section extracts, for the respective plurality of detection areas, heartbeat-related ballistic motion signals each indicating a heartbeat-related ballistic motion of the subject, and the posture determining section determines the posture of the subject by inputting, into a posture determination model, the heartbeat-related ballistic motion signals which have been extracted for the respective detection areas, the posture determination model being a model obtained by machine learning with use of training data in which the heartbeat-related ballistic motion signals, the number of which is the same as the number of the plurality of detection areas, are an explanatory variable and the posture of the subject is a response variable.

With the above configuration, it is possible for the information processing system to determine the posture of a subject more accurately with use of the model obtained by machine learning.

The information processing system in accordance with Aspect 12 of the present disclosure may be configured such that the signal extracting section extracts, from the detection signal, a feature signal including at least one of (i) a heartbeat signal which indicates a heartbeat of the subject, (ii) a breathing vibration signal which indicates a breathing vibration of the subject, (iii) a body motion signal which indicates a body motion of the subject, and (iv) a snore signal which indicates a snore of the subject, the information processing system further includes a sleep determining section that determines a sleep state of the subject on the basis of at least one of the detection signal and the feature signal, and in a case where the sleep determining section determines that the subject is in the sleep state, the peristaltic sound generation determining section determines whether or not the peristaltic sound has been generated.

With the above configuration, the information processing system determines the sleep state of a subject on the basis of at least one of a detection signal and a feature signal. In a case where the information processing system determines that the subject is in the sleep state, the information processing system determines whether or not a peristaltic sound has been generated. In a case where a subject is in a sleep state, there are a few body motions. Therefore, it is possible for the information processing system to accurately determine whether or not a peristaltic sound has been generated.

The information processing system in accordance with Aspect 13 of the present disclosure may be configured such that the sleep determining section determines the sleep state of the subject with use of a sleep determination model obtained by machine learning with use of training data in which a set constituted by, as an element(s), at least one of the detection signal and the feature signal is an explanatory variable and the sleep state of the subject is a response variable, the sleep determining section determining the sleep state of the subject by inputting, into the sleep determination model, a set constituted by an element(s) which is/are at least one of the detection signal that has been outputted from the sensor and the feature signal that has been extracted from the detection signal and which is/are identical to the element(s).

With the above configuration, it is possible for the information processing system to determine the sleep state of a subject more accurately with use of the model obtained by machine learning.

The information processing system in accordance with Aspect 14 of the present disclosure may be configured such that the signal extracting section extracts, from the detection signal, a feature signal including at least one of (i) a heartbeat signal which indicates a heartbeat of the subject, (ii) a breathing vibration signal which indicates a breathing vibration of the subject, (iii) a body motion signal which indicates a body motion of the subject, and (iv) a snore signal which indicates a snore of the subject, the information processing system further includes a sleep determining section that determines a sleep state of the subject on the basis of at least one of the detection signal and the feature signal, and in a case where the sleep determining section determines that the subject is in the sleep state, the generation position estimating section estimates the position of the generation.

With the above configuration, the information processing system determines the sleep state of a subject on the basis of at least one of a detection signal and a feature signal. In a case where the information processing system determines that the subject is in the sleep state, the information processing system estimates the position of generation of a peristaltic sound. In a case where a subject is in a sleep state, there are a few body motions. Therefore, it is possible for the information processing system to accurately estimate the position of generation of a peristaltic sound.

The information processing system in accordance with Aspect 15 of the present disclosure may be configured such that the sleep determining section determines the sleep state of the subject with use of a sleep determination model obtained by machine learning with use of training data in which a set constituted by, as an element(s), at least one of the detection signal and the feature signal is an explanatory variable and the sleep state of the subject is a response variable, the sleep determining section determining the sleep state of the subject by inputting, into the sleep determination model, a set constituted by an element(s) which is/are at least one of the detection signal that has been outputted from the sensor and the feature signal that has been extracted from the detection signal and which is/are identical to the element(s).

With the above configuration, it is possible for the information processing system to determine the sleep state of a subject more accurately with use of the model obtained by machine learning.

The information processing system in accordance with Aspect 16 of the present disclosure may be configured such that the sensor is installed between a bed on which the subject lies and a mattress which is placed on the bed, between a bed sheet which is placed on the mattress and the mattress, on a top surface of the bed, on a backrest of a chair on which the subject sits, or on a seat surface of the chair.

With the above configuration, it is possible for the sensor to detect, from a position at which the sensor does not touch a subject, vibrations which have been generated from the subject. This makes it possible to grasp peristalses of the stomach and intestines without making the subject feel discomfort.

The information processing system in accordance with Aspect 17 of the present disclosure may be configured such that the sensor includes an electrode and a piezoelectric material that generates a voltage by expanding and contracting in a direction perpendicular to a direction in which the electrode extends.

With the above configuration, a reduction in thickness of the sensor is facilitated, and it is therefore possible to reduce the possibility of making a subject feel discomfort.

The information processing system in accordance with Aspect 18 of the present disclosure may be configured such that the piezoelectric material is a foam.

With the above configuration, it is possible for the sensor to detect, with higher accuracy, vibrations generated from a subject.

The information processing system in accordance with Aspect 19 of the present disclosure may be configured such that the sensor has a shape of a thin plate.

With the above configuration, it is possible to install the sensor at various locations. For example, it is also possible to install the sensor on a piece of clothing worn by a subject.

The information processing system in accordance with Aspect 20 of the present disclosure may be configured such that the sensor has a plurality of detection areas that output respective detection signals, the signal extracting section extracts peristaltic sound signals for the respective plurality of detection areas, and the information processing system further includes a contents position estimating section that estimates a position of contents in the stomach and intestines of the subject, on the basis of the peristaltic sound signals which have been extracted, arrangement information which indicates arrangement of the detection areas, and the posture which has been determined.

With the above configuration, the information processing system extracts peristaltic sound signals from detection signals outputted from the respective plurality of detection areas. The information processing system estimates the position of contents in the stomach and intestines on the basis of the peristaltic sound signals which have been extracted, the arrangement information which pertains to the detection areas, and a posture which has been determined.

The information processing system in accordance with Aspect 21 of the present disclosure may be configured such that the contents position estimating section estimates the position of the contents by inputting, into a position estimation model, the peristaltic sound signals which have been extracted, the arrangement information which pertains to the detection areas, and the posture which has been determined, the position estimation model being a model obtained by machine learning with use of training data in which the peristaltic sound signals, the arrangement information which pertains to the detection areas, and the posture of the subject are an explanatory variable and the position of the contents is a response variable.

With the above configuration, it is possible for the information processing system to estimate the position of contents in the stomach and intestines of a subject more accurately with use of the model obtained by machine learning.

The information processing system in accordance with Aspect 22 of the present disclosure may be configured to further include a condition determining section that determines a condition of the stomach and intestines of the subject from the peristaltic sound signal which has been extracted.

With the above configuration, it is possible for the information processing system to determine the condition of the stomach and intestines of a subject which condition corresponds to a peristaltic sound signal that has been extracted. For example, it is possible for a medical expert who refers to the condition of the stomach and intestines which condition has been determined to start a medical intervention on a subject early.

The information processing system in accordance with Aspect 23 of the present disclosure may be configured such that the condition is at least one of ileus, peritonitis, constipation, and diarrhea.

With the above configuration, it is possible to determine, as the condition of the stomach and intestines of a subject, at least one of ileus, peritonitis, constipation, and diarrhea. For example, in the case of functional ileus or peritonitis, no peristaltic sound is not detected. In the case of diarrhea or mechanical ileus, a high-tone peristaltic sound is detected at high frequency. In the case of constipation, a faint and low-tone peristaltic sound is detected.

The information processing system in accordance with Aspect 24 of the present disclosure may be configured such that the condition determining section determines the condition by inputting, into a condition determination model, the peristaltic sound signal which has been extracted, the condition determining model being a model obtained by machine learning with use of training data in which the peristaltic sound signal is an explanatory variable and the condition is a response variable.

With the above configuration, it is possible for the information processing system to determine the condition of the stomach and intestines of a subject more accurately with use of the model obtained by machine learning.

An information processing apparatus in accordance with Aspect 29 of the present disclosure includes: a sensor that is disposed at a given position at which the sensor does not touch a subject and that detects vibrations which have been generated from the subject; a signal extracting section that extracts, from a detection signal outputted from the sensor, a peristaltic sound signal indicating a peristaltic sound in the stomach and intestines of the subject; a posture determining section that determines a posture of the subject on the basis of the detection signal, wherein the signal extracting section extracts the peristaltic sound signal having a signal strength corresponding to the posture which has been determined. This configuration brings about an effect similar to that brought about by the information processing system in accordance with Aspect 1.

An information processing apparatus in accordance with Aspect 30 of the present disclosure includes: a sensor that is disposed at a given position at which the sensor does not touch a subject and that detects vibrations which have been generated from the subject; a signal extracting section that extracts, from a detection signal outputted from the sensor, a heart sound signal indicating a heart sound of the subject and a peristaltic sound signal indicating a peristaltic sound in the stomach and intestines of the subject; and a posture determining section that determines a posture of the subject on the basis of a heartbeat-related ballistic motion signal that is specified from the heart sound signal which has been extracted and that indicates a heartbeat-related ballistic motion, wherein the signal extracting section extracts the peristaltic sound signal having a signal strength corresponding to the posture which has been determined. This configuration brings about an effect similar to that brought about by the information processing system in accordance with Aspect 1.

An information processing apparatus in accordance with Aspect 31 of the present disclosure includes: a posture determining section that determines a posture of a subject on the basis of a detection signal which has been obtained from a signal extracting apparatus including (i) a sensor that is disposed at a given position at which the sensor does not touch the subject and that detects vibrations which have been generated from the subject and (ii) a signal extracting section that extracts a peristaltic sound signal of the subject from the detection signal outputted from the sensor; and a generation position estimating section that estimates a position of generation of a peristaltic sound indicated by the peristaltic sound signal, on the basis of the peristaltic sound signal which has been obtained from the signal extracting apparatus and the posture which has been determined. This configuration brings about an effect similar to that brought about by the information processing system in accordance with Aspect 3.

An information processing apparatus in accordance with Aspect 32 of the present disclosure includes: a posture determining section that determines a posture of a subject on the basis of a heartbeat-related ballistic motion signal which indicates a heartbeat-related ballistic motion and which is specified from a heart sound signal that has been obtained from a signal extracting apparatus including (i) a sensor that is disposed at a given position at which the sensor does not touch the subject and that detects vibrations which have been generated from the subject and (ii) a signal extracting section that extracts, from a detection signal outputted from the sensor, the heart sound signal indicating a heart sound of the subject and a peristaltic sound signal indicating a peristaltic sound in the stomach and intestines of the subject; and a generation position estimating section that estimates a position of generation of the peristaltic sound indicated by the peristaltic sound signal, on the basis of the peristaltic sound signal which has been obtained from the signal extracting apparatus and the posture which has been determined. This configuration brings about an effect similar to that brought about by the information processing system in accordance with Aspect 3.

A control method in accordance with Aspect 33 of the present disclosure is a control method which is carried out by one or more information processing apparatuses, the method including: an outputting step of outputting a detection signal from a sensor that is disposed at a given position at which the sensor does not touch a subject and that detects vibrations which have been generated from the subject; an extracting step of extracting, from the detection signal which has been outputted, a peristaltic sound signal indicating a peristaltic sound in the stomach and intestines of the subject; and a determining step of determining a posture of the subject on the basis of the detection signal, wherein in the extracting step, the peristaltic sound signal having a signal strength corresponding to the posture which has been determined is extracted. This configuration brings about an effect similar to that brought about by the information processing system in accordance with Aspect 1.

A control method in accordance with Aspect 34 of the present disclosure is a control method which is carried out by one or more information processing apparatuses, the method including: an outputting step of outputting a detection signal from a sensor that is disposed at a given position at which the sensor does not touch a subject and that detects vibrations which have been generated from the subject; an extracting step of extracting, from the detection signal which has been outputted, a heart sound signal indicating a heart sound of the subject and a peristaltic sound signal indicating a peristaltic sound in the stomach and intestines of the subject; and a determining step of determining a posture of the subject on the basis of a heartbeat-related ballistic motion signal that is specified from the heart sound signal which has been extracted and that indicates a heartbeat-related ballistic motion, wherein in the extracting step, the peristaltic sound signal having a signal strength corresponding to the posture which has been determined is extracted. This configuration brings about an effect similar to that brought about by the information processing system in accordance with Aspect 1.

A control method in accordance with Aspect 35 of the present disclosure is a control method which is carried out by one or more information processing apparatuses, the method including: an outputting step of outputting a detection signal from a sensor that is disposed at a given position at which the sensor does not touch a subject and that detects vibrations which have been generated from the subject; an extracting step of extracting, from the detection signal which has been outputted, a peristaltic sound signal indicating a peristaltic sound in the stomach and intestines of the subject; a determining step of determining a posture of the subject on the basis of the detection signal; and an estimating step of estimating a position of generation of the peristaltic sound indicated by the peristaltic sound signal, on the basis of the peristaltic sound signal which has been extracted and the posture which has been determined. This configuration brings about an effect similar to that brought about by the information processing system in accordance with Aspect 3.

A control method in accordance with Aspect 36 of the present disclosure is a control method which is carried out by one or more information processing apparatuses, the method including: an outputting step of outputting a detection signal from a sensor that is disposed at a given position at which the sensor does not touch a subject and that detects vibrations which have been generated from the subject; an extracting step of extracting, from the detection signal which has been outputted, a heart sound signal indicating a heart sound of the subject and a peristaltic sound signal indicating a peristaltic sound in the stomach and intestines of the subject; a determining step of determining a posture of the subject on the basis of a heartbeat-related ballistic motion signal that is specified from the heart sound signal which has been extracted and that indicates a heartbeat-related ballistic motion; and an estimating step of estimating a position of generation of the peristaltic sound indicated by the peristaltic sound signal, on the basis of the peristaltic sound signal which has been extracted and the posture which has been determined. This configuration brings about an effect similar to that brought about in Aspect 3.

The information processing systems in accordance with Aspects 1 to 28 of the present invention and the information processing apparatuses in accordance with Aspects 29 to 32 of the present invention may be each implemented by a computer. In this case, the present invention also encompasses, in its scope, (i) a control program for each of the information processing systems and the information processing apparatuses, the control program causing each of the information processing systems and the information processing apparatuses to be implemented by the computer, by causing the computer to operate as each section (software element) included in each of the information processing systems and the information processing apparatuses and (ii) a computer-readable recording medium in which the control program is stored.

A program in accordance with Aspect 37 of the present disclosure is a program for causing a computer to function as the information processing system described in Aspect 1 or 2, the program causing the computer to function as the signal extracting section and the posture determining section.

A program in accordance with Aspect 38 of the present disclosure is a program for causing a computer to function as the information processing system described in Aspect 3 or 4, the program causing the computer to function as the signal extracting section, the posture determining section, and the generation position estimating section.

### [Summary 2]

An information processing system in accordance with an aspect of the present disclosure includes: a sensor that is disposed at a given position at which the sensor does not touch a subject and that detects vibrations which have been generated from the subject; a signal extracting section that extracts, from a detection signal outputted from the sensor, a heart sound signal indicating a heart sound of the subject and a peristaltic sound signal indicating a peristaltic sound in the stomach and intestines of the subject; and a posture determining section that determines a posture of the subject on the basis of a heartbeat-related ballistic motion signal that is specified from the heart sound signal which has been extracted and that indicates a heartbeat-related ballistic motion, wherein the signal extracting section extracts the peristaltic sound signal having a signal strength corresponding to the posture which has been determined.

With the above configuration, the information processing system extracts a heart sound signal and a peristaltic sound signal from a detection signal outputted from the sensor that detects vibrations which have been generated from a subject. The information processing system determines a posture on the basis of a heartbeat-related ballistic motion signal specified from the heart sound signal, and extracts the peristaltic sound signal having a signal strength corresponding to the posture which has been determined.

This makes it possible for the information processing system to extract, in a peristaltic sound signal indicating a peristaltic sound included in vibrations which have been generated from a subject, only the peristaltic sound signal having a signal strength corresponding to the posture of the subject. That is, it is possible to appropriately measure a peristaltic sound regardless of the posture of a subject during measurement.

The information processing system in accordance with an aspect of the present disclosure may be configured to further include a peristaltic sound generation determining section that determines whether or not the peristaltic sound has been generated, wherein the peristaltic sound generation determining section determines that the peristaltic sound has been generated, in a case where the peristaltic sound signal which has been extracted has at least one of (i) a given frequency characteristic and (ii) a given signal strength at a given frequency.

With the above configuration, the information processing system determines whether or not a peristaltic sound has been generated, on the basis of at least one of the frequency characteristic and the signal strength of a peristaltic sound signal of a subject. This makes it possible for the information processing system to accurately determine a peristaltic sound included in vibrations which have been generated from a subject.

An information processing system in accordance with an aspect of the present disclosure includes: a sensor that is disposed at a given position at which the sensor does not touch a subject and that detects vibrations which have been generated from the subject; a signal extracting section that extracts, from a detection signal outputted from the sensor, a heart sound signal indicating a heart sound of the subject and a peristaltic sound signal indicating a peristaltic sound in the stomach and intestines of the subject; a posture determining section that determines a posture of the subject on the basis of a heartbeat-related ballistic motion signal that is specified from the heart sound signal which has been extracted and that indicates a heartbeat-related ballistic motion; and a generation position estimating section that estimates a position of generation of the peristaltic sound indicated by the peristaltic sound signal, on the basis of the peristaltic sound signal which has been extracted and the posture which has been determined.

With the above configuration, the information processing system extracts a heart sound signal and a peristaltic sound signal from a detection signal outputted from the sensor that detects vibrations which have been generated from a subject. The information processing system determines a posture on the basis of the heartbeat-related ballistic motion signal specified from the heart sound signal, and estimates the position of generation of a peristaltic sound on the basis of the peristaltic sound signal which has been extracted and the posture which has been determined.

This makes it possible for the information processing system to estimate the position of generation of a peristaltic sound included in vibrations which have been generated from a subject. For example, in a case where a peristaltic sound signal which has been extracted indicates an abnormality in the stomach and intestines, it is possible to for a medical expert to provide an appropriate treatment, considering that the abnormality is occurring at the position of generation of a peristaltic sound.

The information processing system in accordance with an aspect of the present disclosure may be configured such that the signal extracting section extracts, from the detection signal as the peristaltic sound signal, at least one of (i) a signal having a peak in 150 Hz to 300 Hz and (ii) a signal having a peak in 500 Hz to 700 Hz.

With the above configuration, it is possible for the information processing system to extract a peristaltic sound signal from a detection signal on the basis of a frequency characteristic.

The information processing system in accordance with an aspect of the present disclosure may be configured such that the posture determining section determines the posture of the subject by inputting, into a posture determination model, the heartbeat-related ballistic motion signal which has been specified, the posture determination model being a model obtained by machine learning with use of training data in which the heartbeat-related ballistic motion signal is an explanatory variable and the posture of the subject is a response variable.

With the above configuration, it is possible for the information processing system to determine the posture of a subject more accurately by using the model obtained by machine learning.

The information processing system in accordance with an aspect of the present disclosure may be configured such that the sensor has a plurality of detection areas that output respective detection signals, the signal extracting section extracts, for the respective plurality of detection areas, heartbeat-related ballistic motion signals each indicating a heartbeat-related ballistic motion of the subject, and the posture determining section determines the posture of the subject on the basis of a result of comparison of frequency characteristics and/or signal strengths of the heartbeat-related ballistic motion signals which have been extracted for the respective detection areas.

With the above configuration, the information processing system extracts heartbeat-related ballistic motion signals from detection signals outputted from the respective plurality of detection areas, and determines the posture of a subject on the basis of the frequency characteristics and/or the signal strengths of the heartbeat-related ballistic motion signals which have been extracted for the respective plurality of detection areas. This makes it possible for the information processing system to more accurately determine the posture of a subject at the time when the sensor has detected vibrations generated from the subject.

The information processing system in accordance with an aspect of the present disclosure may be configured such that the sensor has a plurality of detection areas that output respective detection signals, the signal extracting section extracts heartbeat-related ballistic motion signals each indicating a heartbeat-related ballistic motion of the subject, and the posture determining section determines the posture of the subject by inputting, into a posture determination model, the heartbeat-related ballistic motion signals specified from heart sound signals which have been extracted for the respective detection areas, the posture determination model being a model obtained by machine learning with use of training data in which the heartbeat-related ballistic motion signals, the number of which is the same as the number of the plurality of detection areas, are an explanatory variable and the posture of the subject is a response variable.

With the above configuration, it is possible for the information processing system to determine the posture of a subject more accurately with use of the model obtained by machine learning.

The information processing system in accordance with an aspect of the present disclosure may be configured such that the signal extracting section extracts, from the detection signal, a feature signal including at least one of (i) a heartbeat signal which indicates a heartbeat of the subject, (ii) a breathing vibration signal which indicates a breathing vibration of the subject, (iii) a body motion signal which indicates a body motion of the subject, and (iv) a snore signal which indicates a snore of the subject, the information processing system further includes a sleep determining section that determines a sleep state of the subject on the basis of at least one of the detection signal and the feature signal, and in a case where the sleep determining section determines that the subject is in the sleep state, the peristaltic sound generation determining section determines whether or not the peristaltic sound has been generated.

With the above configuration, the information processing system determines the sleep state of a subject on the basis of at least one of a detection signal and a feature signal. In a case where the information processing system determines that the subject is in the sleep state, the information processing system determines whether or not a peristaltic sound has been generated. In a case where a subject is in a sleep state, there are a few body motions. Therefore, it is possible for the information processing system to accurately determine whether or not a peristaltic sound has been generated.

The information processing system in accordance with an aspect of the present disclosure may be configured such that the signal extracting section extracts, from the detection signal, a feature signal including at least one of (i) a heartbeat signal which indicates a heartbeat of the subject, (ii) a breathing vibration signal which indicates a breathing vibration of the subject, (iii) a body motion signal which indicates a body motion of the subject, and (iv) a snore signal which indicates a snore of the subject, the information processing system further includes a sleep determining section that determines a sleep state of the subject on the basis of at least one of the detection signal and the feature signal, and in a case where the sleep determining section determines that the subject is in the sleep state, the generation position estimating section estimates the position of the generation.

With the above configuration, the information processing system determines the sleep state of a subject on the basis of at least one of a detection signal and a feature signal. In a case where it is determined that the subject is in the sleep state, the information processing system estimates the position of generation of a peristaltic sound. In a case where a subject is in a sleep state, there are a few body motions. Therefore, it is possible for the information processing system to accurately estimate the position of generation of a peristaltic sound.

The information processing system in accordance with an aspect of the present disclosure may be configured such that the sleep determining section determines the sleep state of the subject with use of a sleep determination model obtained by machine learning with use of training data in which a set constituted by, as an element(s), at least one of the detection signal and the feature signal is an explanatory variable and the sleep state of the subject is a response variable, the sleep determining section determining the sleep state of the subject by inputting, into the sleep determination model, a set constituted by an element(s) which is/are at least one of the detection signal that has been outputted from the sensor and the feature signal that has been extracted from the detection signal and which is/are identical to the element(s).

With the above configuration, it is possible for the information processing system to determine the sleep state of a subject more accurately with use of the model obtained by machine learning.

The information processing system in accordance with an aspect of the present disclosure may be configured such that the sensor is installed between a bed on which the subject lies and a mattress which is placed on the bed, between a bed sheet which is placed on the mattress and the mattress, on a top surface of the bed, on a backrest of a chair on which the subject sits, or on a seat surface of the chair.

With the above configuration, it is possible for the sensor to detect, from a position at which the sensor does not touch a subject, vibrations which have been generated from the subject. This makes it possible to grasp peristalses of the stomach and intestines without making the subject feel discomfort.

The information processing system in accordance with an aspect of the present disclosure may be configured such that the sensor includes an electrode and a piezoelectric material that generates a voltage by expanding and contracting in a direction perpendicular to a direction in which the electrode extends.

With the above configuration, a reduction in thickness of the sensor is facilitated, and it is therefore possible to reduce the possibility of making a subject feel discomfort.

The information processing system in accordance with an aspect of the present disclosure may be configured such that the piezoelectric material is a foam.

With the above configuration, it is possible for the sensor to detect, with higher accuracy, vibrations generated from a subject.

The information processing system in accordance with an aspect of the present disclosure may be configured such that the sensor has a shape of a thin plate.

With the above configuration, it is possible to install the sensor at various locations. For example, it is also possible to install the sensor on a piece of clothing worn by a subject.

The information processing system in accordance with an aspect of the present disclosure may be configured such that the sensor has a plurality of detection areas that output respective detection signals, the signal extracting section extracts peristaltic sound signals for the respective plurality of detection areas, and the information processing system further includes a contents position estimating section that estimates a position of contents in the stomach and intestines of the subject, on the basis of the peristaltic sound signals which have been extracted, arrangement information which indicates arrangement of the detection areas, and the posture which has been determined.

With the above configuration, the information processing system extracts peristaltic sound signals from detection signals outputted from the respective plurality of detection areas. The information processing system estimates the position of contents in the stomach and intestines on the basis of the peristaltic sound signals which have been extracted, the arrangement information which pertains to the detection areas, and a posture which has been determined.

The information processing system in accordance with an aspect of the present disclosure may be configured such that the contents position estimating section estimates the position of the contents by inputting, into a position estimation model, the peristaltic sound signals which have been extracted, the arrangement information which pertains to the detection areas, and the posture which has been determined, the position estimation model being a model obtained by machine learning with use of training data in which the peristaltic sound signals, the arrangement information which pertains to the detection areas, and the posture of the subject are an explanatory variable and the position of the contents is a response variable.

With the above configuration, it is possible for the information processing system to estimate the position of contents in the stomach and intestines of a subject more accurately with use of the model obtained by machine learning.

The information processing system in accordance with an aspect of the present disclosure may be configured to further include a condition determining section that determines a condition of the stomach and intestines of the subject from the peristaltic sound signal which has been extracted.

With the above configuration, it is possible for the information processing system to determine the condition of the stomach and intestines of a subject which condition corresponds to a peristaltic sound signal that has been extracted. For example, it is possible for a medical expert who refers to the condition of the stomach and intestines which condition has been determined to start a medical intervention on a subject early.

The information processing system in accordance with an aspect of the present disclosure may be configured such that the condition is at least one of ileus, peritonitis, constipation, and diarrhea.

With the above configuration, it is possible to determine, as the condition of the stomach and intestines of a subject, at least one of ileus, peritonitis, constipation, and diarrhea. For example, in the case of functional ileus or peritonitis, no peristaltic sound is not detected. In the case of diarrhea or mechanical ileus, a high-tone peristaltic sound is detected at high frequency. In the case of constipation, a faint and low-tone peristaltic sound is detected.

The information processing system in accordance with an aspect of the present disclosure may be configured such that the condition determining section determines the condition by inputting, into a condition determination model, the peristaltic sound signal which has been extracted, the condition determining model being a model obtained by machine learning with use of training data in which the peristaltic sound signal is an explanatory variable and the condition is a response variable.

With the above configuration, it is possible for the information processing system to determine the condition of the stomach and intestines of a subject more accurately with use of the model obtained by machine learning.

An information processing apparatus in accordance with an aspect of the present disclosure includes: a sensor that is disposed at a given position at which the sensor does not touch a subject and that detects vibrations which have been generated from the subject; a signal extracting section that extracts, from a detection signal outputted from the sensor, a heart sound signal indicating a heart sound of the subject and a peristaltic sound signal indicating a peristaltic sound in the stomach and intestines of the subject; and a posture determining section that determines a posture of the subject on the basis of a heartbeat-related ballistic motion signal that is specified from the heart sound signal which has been extracted and that indicates a heartbeat-related ballistic motion, wherein the signal extracting section extracts the peristaltic sound signal having a signal strength corresponding to the posture which has been determined. This configuration brings about an effect similar to that brought about by the information processing system in accordance with Aspect 1.

An information processing apparatus in accordance with an aspect of the present disclosure includes: a posture determining section that determines a posture of a subject on the basis of a heartbeat-related ballistic motion signal which indicates a heartbeat-related ballistic motion and which is specified from a heart sound signal that has been obtained from a signal extracting apparatus including (i) a sensor that is disposed at a given position at which the sensor does not touch the subject and that detects vibrations which have been generated from the subject and (ii) a signal extracting section that extracts, from a detection signal outputted from the sensor, the heart sound signal indicating a heart sound of the subject and a peristaltic sound signal indicating a peristaltic sound in the stomach and intestines of the subject; and a generation position estimating section that estimates a position of generation of the peristaltic sound indicated by the peristaltic sound signal, on the basis of the peristaltic sound signal which has been obtained from the signal extracting apparatus and the posture which has been determined. This configuration brings about an effect similar to that brought about by the information processing system in accordance with Aspect 3.

A control method in accordance with an aspect of the present disclosure is a control method which is carried out by one or more information processing apparatuses, the method including: an outputting step of outputting a detection signal from a sensor that is disposed at a given position at which the sensor does not touch a subject and that detects vibrations which have been generated from the subject; an extracting step of extracting, from the detection signal which has been outputted, a heart sound signal indicating a heart sound of the subject and a peristaltic sound signal indicating a peristaltic sound in the stomach and intestines of the subject; and a determining step of determining a posture of the subject on the basis of a heartbeat-related ballistic motion signal that is specified from the heart sound signal which has been extracted and that indicates a heartbeat-related ballistic motion, wherein in the extracting step, the peristaltic sound signal having a signal strength corresponding to the posture which has been determined is extracted. This makes it possible for the information processing system to extract, in a peristaltic sound signal indicating a peristaltic sound included in vibrations which have been generated from a subject, only the peristaltic sound signal having a signal strength corresponding to the posture of the subject. That is, it is possible to appropriately measure a peristaltic sound regardless of the posture of a subject during measurement.

A control method in accordance with an aspect of the present disclosure is a control method which is carried out by one or more information processing apparatuses, the method including: an outputting step of outputting a detection signal from a sensor that is disposed at a given position at which the sensor does not touch a subject and that detects vibrations which have been generated from the subject; an extracting step of extracting, from the detection signal which has been outputted, a heart sound signal indicating a heart sound of the subject and a peristaltic sound signal indicating a peristaltic sound in the stomach and intestines of the subject; a determining step of determining a posture of the subject on the basis of a heartbeat-related ballistic motion signal that is specified from the heart sound signal which has been extracted and that indicates a heartbeat-related ballistic motion; and an estimating step of estimating a position of generation of the peristaltic sound indicated by the peristaltic sound signal, on the basis of the peristaltic sound signal which has been extracted and the posture which has been determined. This makes it possible for an information processing system to estimate the position of generation of a peristaltic sound included in vibrations which have been generated from a subject. For example, in a case where a peristaltic sound signal which has been extracted indicates an abnormality in the stomach and intestines, it is possible to for a medical expert to provide an appropriate treatment, considering that the abnormality is occurring at the position of generation of a peristaltic sound.

The information processing systems in accordance with aspects of the present disclosure and the information processing apparatuses in accordance with aspects of the present disclosure may be each implemented by a computer. In this case, the present invention also encompasses, in its scope, (i) a control program for each of the information processing systems and the information processing apparatuses, the control program causing each of the information processing systems and the information processing apparatuses to be implemented by the computer, by causing the computer to operate as each section (software element) included in each of the information processing systems and the information processing apparatuses and (ii) a computer-readable recording medium in which the control program is stored.

### [Summary 3]

An information processing system in accordance with Aspect 1 of the present disclosure includes: a sensor that is disposed at a given position at which the sensor does not touch a subject and that detects vibrations which have been generated from the subject; and a peristaltic sound generation determining section that determines whether or not a peristaltic sound has been generated in the stomach and intestines of the subject, on the basis of a detection signal outputted from the sensor.

An information processing system in accordance with Aspect 2 of the present disclosure includes: a sensor that is disposed at a given position at which the sensor does not touch a subject and that detects vibrations which have been generated from the subject; and a signal extracting section that extracts a peristaltic sound signal indicating a peristaltic sound in the stomach and intestines of the subject, on the basis of a detection signal outputted from the sensor.

The information processing system in accordance with Aspect 3 of the present disclosure may be configured to, in Aspect 2, further include a posture determining section that determines a posture of the subject, wherein the signal extracting section extracts the peristaltic sound signal corresponding to the posture which has been determined.

An information processing system in accordance with Aspect 4 of the present disclosure includes: a sensor that is disposed at a given position at which the sensor does not touch a subject and that detects vibrations which have been generated from the subject; a signal extracting section that extracts, from a detection signal outputted from the sensor, a peristaltic sound signal indicating a peristaltic sound in the stomach and intestines of the subject; and a posture determining section that determines a posture of the subject on the basis of the detection signal, wherein the signal extracting section extracts the peristaltic sound signal corresponding to the posture which has been determined.

An information processing system in accordance with Aspect 5 of the present disclosure includes: a sensor that is disposed at a given position at which the sensor does not touch a subject and that detects vibrations which have been generated from the subject; a signal extracting section that extracts, from a detection signal outputted from the sensor, a heart sound signal indicating a heart sound of the subject and a peristaltic sound signal indicating a peristaltic sound in the stomach and intestines of the subject; and a posture determining section that determines a posture of the subject on the basis of the heart sound signal which has been extracted, wherein the signal extracting section extracts the peristaltic sound signal having a signal strength corresponding to the posture which has been determined.

An information processing system in accordance with Aspect 6 of the present disclosure includes: a sensor that is disposed at a given position at which the sensor does not touch a subject and that detects vibrations which have been generated from the subject; a signal extracting section that extracts, from a detection signal outputted from the sensor, a peristaltic sound signal indicating a peristaltic sound in the stomach and intestines of the subject; a posture determining section that determines a posture of the subject; and a generation position estimating section that estimates a position of generation of the peristaltic sound indicated by the peristaltic sound signal, on the basis of the peristaltic sound signal which has been extracted and the posture which has been determined.

The information processing system in accordance with an aspect of the present disclosure may be configured such that, in any one of Aspects 3 through 5, the sensor has a plurality of detection areas that output respective detection signals, and the posture determining section determines the posture of the subject on the basis of a result of comparison of signal strengths of the detection signals that have been outputted from the respective plurality of detection areas.

The information processing system in accordance with Aspect 8 of the present disclosure may be configured such that, in any one of Aspects 3 through 6, the sensor has a plurality of detection areas that output respective detection signals, the signal extracting section extracts, for the respective plurality of detection areas, heart sound signals each indicating a heart sound of the subject, and the posture determining section determines the posture of the subject on the basis of a result of comparison of frequency characteristics and/or signal strengths of the heart sound signals which have been extracted for the respective plurality of detection areas.

The information processing system in accordance with Aspect 9 of the present disclosure may be configured to, in Aspect 1, further include a sleep determining section that determines a sleep state of the subject, wherein in a case where the sleep determining section determines that the subject is in the sleep state, the peristaltic sound generation determining section determines whether or not the peristaltic sound has been generated.

The information processing system in accordance with Aspect 10 of the present disclosure may be configured to, in Aspect 1 or 9, further include a signal extracting section that extracts a peristaltic sound signal indicating the peristaltic sound in the stomach and intestines of the subject, on the basis of the detection signal outputted from the sensor, wherein the signal extracting section extracts, from the detection signal, a feature signal including at least one of (i) a heartbeat signal which indicates a heartbeat of the subject, (ii) a breathing vibration signal which indicates a breathing vibration of the subject, (iii) a body motion signal which indicates a body motion of the subject, and (iv) a snore signal which indicates a snore of the subject, the information processing system further includes a sleep determining section that determines a sleep state of the subject on the basis of at least one of the detection signal and the feature signal, and in a case where the sleep determining section determines that the subject is in the sleep state, the peristaltic sound generation determining section determines whether or not the peristaltic sound has been generated.

The information processing system in accordance with Aspect 11 of the present disclosure may be configured such that, in Aspect 6, the signal extracting section extracts, from the detection signal, a feature signal including at least one of (i) a heartbeat signal which indicates a heartbeat of the subject, (ii) a breathing vibration signal which indicates a breathing vibration of the subject, (iii) a body motion signal which indicates a body motion of the subject, and (iv) a snore signal which indicates a snore of the subject, the information processing system further includes a sleep determining section that determines a sleep state of the subject on the basis of at least one of the detection signal and the feature signal, and in a case where the sleep determining section determines that the subject is in the sleep state, the generation position estimating section estimates the position of the generation.

The information processing system in accordance with Aspect 12 of the present disclosure may be configured such that, in any one of Aspects 1 through 11, the sensor is installed between a bed on which the subject lies and a mattress which is placed on the bed, between a bed sheet which is placed on the mattress and the mattress, on a top surface of the bed, on a backrest of a chair on which the subject sits, or on a seat surface of the chair.

The information processing system in accordance with Aspect 13 of the present disclosure may be configured such that, in any one of Aspects 1 through 12, the sensor includes an electrode and a piezoelectric material that generates a voltage by expanding and contracting in a direction perpendicular to a direction in which the electrode extends.

The information processing system in accordance with Aspect 14 of the present disclosure may be configured such that the piezoelectric material is a foam.

The information processing system in accordance with Aspect 15 of the present disclosure may be configured such that, in any one of Aspects 1 through 14, the sensor has a shape of a thin plate.

The information processing system in accordance with Aspect 16 of the present disclosure may be configured to, in any one of Aspects 1 through 15, further include a signal extracting section that extracts a peristaltic sound signal indicating the peristaltic sound in the stomach and intestines of the subject, on the basis of the detection signal outputted from the sensor, wherein the sensor has a plurality of detection areas that output respective detection signals, the signal extracting section extracts peristaltic sound signals for the respective plurality of detection areas, and the information processing system further includes a contents position estimating section that estimates a position of contents in the stomach and intestines of the subject, on the basis of the peristaltic sound signals which have been extracted, arrangement information which indicates arrangement of the plurality of detection areas, and a posture which has been determined.

The information processing system in accordance with Aspect 17 of the present disclosure may be configured to, in any one of Aspects 1 through 16, further include a condition determining section that determines a condition of the stomach and intestines of the subject from a peristaltic sound signal which has been extracted.

The information processing system in accordance with Aspect 18 of the present disclosure may be configured such that, in Aspect 17, the condition is at least one of ileus, peritonitis, constipation, and diarrhea.

The information processing system in accordance with Aspect 19 of the present disclosure may be configured such that, in Aspect 17 or 18, the condition determining section determines the condition by inputting, into a condition determination model, the peristaltic sound signal which has been extracted, the condition determining model being a model obtained by machine learning with use of training data in which the peristaltic sound signal is an explanatory variable and the condition is a response variable.

An information processing apparatus in accordance with Aspect 20 of the present disclosure includes: a sensor that is disposed at a given position at which the sensor does not touch a subject and that detects vibrations which have been generated from the subject; and a signal extracting section that extracts, from a detection signal outputted from the sensor, a peristaltic sound signal indicating a peristaltic sound in the stomach and intestines of the subject.

A control method in accordance with Aspect 21 of the present disclosure is a control method which is carried out by one or more information processing apparatuses, the method including: an outputting step of outputting a detection signal from a sensor that is disposed at a given position at which the sensor does not touch a subject and that detects vibrations which have been generated from the subject; an extracting step of extracting, from the detection signal which has been outputted, a peristaltic sound signal indicating a peristaltic sound in the stomach and intestines of the subject; and a determining step of determining a posture of the subject on the basis of the detection signal.

A program in accordance with Aspect 22 of the present disclosure is a program for causing a computer to function as the information processing system described in Aspect 1, the program causing the computer to function as the peristaltic sound generation determining section.

A program in accordance with Aspect 23 of the present disclosure is a program for causing a computer to function as the information processing system described in Aspect 2, the program causing the computer to function as the signal extracting section.

### REFERENCE SIGNS LIST

100, 100a, 100b, 100c, 100d, 200, 200a, 200b, 200c, 400, 400a, 400b, 400c, 400d, 500, 500a, 500b, 500c Information processing system
1, 1a, 1b, 1c, 1d, 2, 2a, 2b, 2c, 4, 4a, 4b, 4c, 4d, 5, 5a, 5b, 5c Information processing apparatus
3 Communication apparatus
11 Sensor
121, 121b, 221, 421, 421b, 521, 521a Signal extracting section
122, 422, 522 Posture determining section
123, 423, 523 Output section
124a, 424a Peristaltic sound generation determining section
125b, 225, 425b, 525a Sleep determining section
126c, 226, 426c, 526b Condition determining section
127d, 227, 427d, 527c Contents position estimating section

1. An information processing system comprising:
a sensor that is disposed at a given position at which the sensor does not touch a subject and that detects vibrations which have been generated from the subject; and
a signal extracting section that extracts a peristaltic sound signal indicating a peristaltic sound in the stomach and intestines of the subject, on the basis of a detection signal outputted from the sensor.

2. (canceled)

3. The information processing system as set forth in claim 1, further comprising
a posture determining section that determines a posture of the subject on the basis of the detection signal.

4. An information processing system as set forth in claim 3, wherein
the signal extracting section extracts the peristaltic sound signal corresponding to the posture which has been determined.

5. An information processing system as set forth in claim 3, wherein
the signal extracting section further extracts a heart sound signal indicating a heart sound of the subject from the detection signal outputted from the sensor,
the posture determining section determines a posture of the subject on the basis of the heart sound signal which has been extracted, and
the signal extracting section extracts the peristaltic sound signal having a signal strength corresponding to the posture which has been determined.

6. An information processing system as set forth in claim 3, further comprising:
a generation position estimating section that estimates a position of generation of the peristaltic sound indicated by the peristaltic sound signal, on the basis of the peristaltic sound signal which has been extracted and the posture which has been determined.

7. The information processing system as set forth in claim 3, wherein
the sensor has a plurality of detection areas that output respective detection signals, and
the posture determining section determines the posture of the subject on the basis of a result of comparison of signal strengths of the detection signals that have been outputted from the respective plurality of detection areas.

8. The information processing system as set forth in claim 3, wherein
the sensor has a plurality of detection areas that output respective detection signals,
the signal extracting section extracts, for the respective plurality of detection areas, heart sound signals each indicating a heart sound of the subject, and
the posture determining section determines the posture of the subject on the basis of a result of comparison of frequency characteristics and/or signal strengths of the heart sound signals which have been extracted for the respective plurality of detection areas.

9. The information processing system as set forth in claim 1, further comprising
a sleep determining section that determines a sleep state of the subject, wherein
in a case where the sleep determining section determines that the subject is in the sleep state, the peristaltic sound generation determining section determines whether or not the peristaltic sound has been generated.

10. The information processing system as set forth in claim 1, further comprising
a signal extracting section that extracts a peristaltic sound signal indicating the peristaltic sound in the stomach and intestines of the subject, on the basis of the detection signal outputted from the sensor, wherein
the signal extracting section extracts, from the detection signal, a feature signal including at least one of (i) a heartbeat signal which indicates a heartbeat of the subject, (ii) a breathing vibration signal which indicates a breathing vibration of the subject, (iii) a body motion signal which indicates a body motion of the subject, and (iv) a snore signal which indicates a snore of the subject,
said information processing system further comprises a sleep determining section that determines a sleep state of the subject on the basis of at least one of the detection signal and the feature signal, and
in a case where the sleep determining section determines that the subject is in the sleep state, the peristaltic sound generation determining section determines whether or not the peristaltic sound has been generated.

11. The information processing system as set forth in claim 6, wherein
the signal extracting section extracts, from the detection signal, a feature signal including at least one of (i) a heartbeat signal which indicates a heartbeat of the subject, (ii) a breathing vibration signal which indicates a breathing vibration of the subject, (iii) a body motion signal which indicates a body motion of the subject, and (iv) a snore signal which indicates a snore of the subject,
said information processing system further comprises a sleep determining section that determines a sleep state of the subject on the basis of at least one of the detection signal and the feature signal, and
in a case where the sleep determining section determines that the subject is in the sleep state, the generation position estimating section estimates the position of the generation.

12. The information processing system as set forth in claim 1, wherein
the sensor is installed
between a bed on which the subject lies and a mattress which is placed on the bed,
between a bed sheet which is placed on the mattress and the mattress,
on a top surface of the bed,
on a backrest of a chair on which the subject sits, or
on a seat surface of the chair.

13. The information processing system as set forth in claim 1, wherein
the sensor has a shape of a thin plate,
the sensor includes an electrode and a piezoelectric material that generates a voltage by expanding and contracting in a direction perpendicular to a direction in which the electrode extends, and
the piezoelectric material is a foam.

14. (canceled)

15. (canceled)

16. The information processing system as set forth in claim 1, further comprising
a signal extracting section that extracts a peristaltic sound signal indicating the peristaltic sound in the stomach and intestines of the subject, on the basis of the detection signal outputted from the sensor, wherein
the sensor has a plurality of detection areas that output respective detection signals,
the signal extracting section extracts peristaltic sound signals for the respective plurality of detection areas, and
said information processing system further comprises a contents position estimating section that estimates a position of contents in the stomach and intestines of the subject, on the basis of the peristaltic sound signals which have been extracted, arrangement information which indicates arrangement of the plurality of detection areas, and a posture which has been determined.

17. The information processing system as set forth in claim 1, further comprising
a condition determining section that determines a condition of the stomach and intestines of the subject from a peristaltic sound signal which has been extracted.

18. The information processing system as set forth in claim 17, wherein
the condition is at least one of ileus, peritonitis, constipation, and diarrhea.

19. The information processing system as set forth in claim 17, wherein
the condition determining section determines the condition by inputting, into a condition determination model, the peristaltic sound signal which has been extracted, the condition determining model being a model obtained by machine learning with use of training data in which the peristaltic sound signal is an explanatory variable and the condition is a response variable.

20. An information processing apparatus comprising:
a sensor that is disposed at a given position at which the sensor does not touch a subject and that detects vibrations which have been generated from the subject; and
a signal extracting section that extracts, from a detection signal outputted from the sensor, a peristaltic sound signal indicating a peristaltic sound in the stomach and intestines of the subject.

21. A control method which is carried out by one or more information processing apparatuses, said method comprising:
an outputting step of outputting a detection signal from a sensor that is disposed at a given position at which the sensor does not touch a subject and that detects vibrations which have been generated from the subject;
an extracting step of extracting, from the detection signal which has been outputted, a peristaltic sound signal indicating a peristaltic sound in the stomach and intestines of the subject; and
a determining step of determining a posture of the subject on the basis of the detection signal.

22. A non-transitory computer-readable storage medium storing a computer program for causing a computer to function as the information processing system recited in claim 1, the computer program causing the computer to function as the peristaltic sound generation determining section by executing the computer program with a processor of the computer.

23. A non-transitory computer-readable storage medium storing a computer program for causing a computer to function as the information processing system recited in claim 3, the computer program causing the computer to function as the signal extracting section by executing the computer program with a processor of the computer.
